(19) United States
(12) Patent Application Publication
(10) Pub. No.: US 2016/0030300 A1
(43) Pub. Date: Feb. 4, 2016

(54) Multi-Step Regimen For Improving The Appearance And Feel Of Human Skin

(71) Applicant: The Procter & Gamble Company, Cincinnati, OH (US)

(72) Inventors: Joseph HARRY JANSEN, Harrison, OH (US); Paul Robert TANNER, Lebanon, OH (US)

(21) Appl. No.: 14/445,434

(22) Filed: Jul. 29, 2014

(51) Int. Cl. A61K 8/02; A61Q 17/04

(52) U.S. Cl. CPC A61K 8/025; A61Q 17/04

(57) ABSTRACT

A method of improving the look and feel of skin by using a two-step regimen that includes applying an underlying layer of a skin care composition and applying an overlying layer of a finisher composition on top of the underlying skin care composition layer. The finisher composition is an oil-in-water emulsion comprising a continuous aqueous phase, a dispersed oil phase, and from 10 to 25 wt % of substantially spherical particles. The oil phase includes non-volatile oil that is at least 50 wt % of a liquid UV agent. The spherical particles can be starch particles, silicone elastomer particles or combinations of these. The finisher composition is generally free of glycerin or other humectants.

### FIELD OF THE INVENTION

The present disclosure relates generally to a multi-step method of improving the appearance and feel of human skin. More specifically, the method includes applying a finisher composition that has a high powder content and a UV agent as an overlying top layer to an underlying layer of skin care composition, thereby improving the look and feel of the treated skin.

### BACKGROUND OF THE INVENTION

Personal care products are well known and widely used. These products have long been employed to protect, cleanse and moisturize, deliver actives, hide imperfections and reduce the oiliness and shine on keratinous surfaces. Personal care products have also been used to alter the color and appearance of skin and hair. A variety of personal-care compositions are available to provide skin care benefits and to help prevent and even counteract what many consumers consider to be undesirable signs of skin aging (e.g., fine lines, wrinkles, and uneven skin texture). Of these benefits, look and feel are generally considered to be the two most important and desired effects by consumers.

Traditionally, a wide variety of different functional materials are combined in a single skin care product in an attempt to deliver a range of look and/or feel benefits to consumers. For example, a conventional skin care product might contain skin care actives such as humectants to improve the condition and health of the skin, emollients to lubricate the skin, powders to provide a skin feel and immediate skin appearance benefit, and/or UV actives to absorb damaging ultraviolet radiation (“UV”). But combining some or all of these compositions into one product can have difficulties.

Moreover, products that deliver one benefit are generally intended as a single application product. Layering several products on the skin, which deliver different benefits but are intended to be used individually, may have some of the same drawbacks and complications as mixing too many ingredients in one composition. Hence there is a need to deliver improved look and feel benefits while minimizing the undesirable tradeoffs associated with using multiple single-application products.

For example, particulate material can be added to consumer products for a variety of reasons such as to improve the skin feel of the product. Particulate materials also may provide an immediate visible benefit to the skin by diffusely reflecting light, which provides a matting effect to the skin. However, many particulate materials are added to act as an opacifying agent, which effectively turns the consumer product into make-up or make-up like product. The acute and chronic benefits of personal care compositions having opacifying agents are often lost on the user who only appreciates the masking effect that the opacifying agents provide. Examples of these include high refractive index pigments, such as titanium dioxide and iron oxides, to provide skin color benefits.

Micronized or spherical polymer particles may be used to provide feel and visible texture, wrinkle reduction benefits. For these materials, however, there are tradeoffs if one attempts to increase these feel and look benefits. Using high levels of powder typically leads to products that are hard to spread on skin, and that lose their look benefits over time. Such products can become noticeably white and can flake off the skin.

Another group of ingredients known to provide a multitude of skin health and appearance benefits are humectants. Humectants may increase skin translucency, as evidenced by less surface scattering and reducing refractive index gradients in the stratum corneum; reduce visible texture (i.e., plumping of the stratum corneum); and generally improve skin function and strength. Glycerin is one of the most efficient humectants available due to its chemical structure. But glycerin is a very thick, sticky material and high levels of glycerin can feel very sticky and heavy on the skin. Moreover, high levels of glycerin on the skin can make it look very shiny and greasy, given that glycerin is slow to absorb into the skin.

UV agents are also well known ingredients in skin care compositions and impart a film that protects skin against the damaging effects of exposure to ultraviolet radiation from the sun. UV agents absorb and/or diffuse UV before it can interact with and damage skin. The ability of a composition to protect skin from UV is usually expressed as a sun protection factor (“SPF”) rating. Combinations of UV agents are typically used in personal care compositions in order to raise the SPF rating of the composition and to offer broad spectrum protection for damaging UV (e.g., UV-A and UV-B). However, many UV agents (e.g., liquid UV agents and oil-soluble solid UV agents) are oily materials. Thus, while the ability of a composition to protect skin from UV may be improved by increasing the amount of UV agents, the resulting composition may have a heavy oily skin feel.

Therefore, a need exists for personal care compositions that deliver suitable levels of look and feel benefits with UV protection when used together while minimizing the undesirable tradeoffs associated with using multiple single-application cosmetic products.

### SUMMARY OF THE INVENTION

In order to overcome the problems of conventional skin care products, disclosed herein is a method of improving the look and feel of skin. The method comprises applying an underlying layer of a skin care composition to a target portion of skin, and applying an overlying layer of a finisher composition on top of the underlying skin care composition layer. The finisher composition is an oil-in-water emulsion comprising a continuous aqueous phase, a dispersed oil phase, and from 10 to 25 wt % of substantially spherical particles. The aqueous phase includes water at about 20 to 85% by weight of the finisher composition. The oil phase includes non-volatile oil. The non-volatile oil includes at least 50 wt % of a liquid UV agent, based on the weight of the non-volatile oil. The spherical particles are selected from the group consisting of starch particles, silicone elastomer particles and combinations thereof, and have a particle size of from about 2 to 40 microns. The weight ratio of the non-volatile oil to the particles is from about 1:10 to about 1:1. Optionally, the compositions herein may include from about 1 to 20 wt % of a volatile oil. And in some embodiments, the finisher composition is free or substantially free of glycerin or other humectants.

### DETAILED DESCRIPTION

The methods, regimens and compositions herein overcome at least some of the issues of prior personal care compositions and regimens. By placing different skin care components in different compositions and then applying them in a particular order in a multi step process, the sticky, heavy feel of the humectants is greatly reduced, the smooth, powdery feel of the powders is enhanced, and the appearance benefits from the powders is significantly increased. This layering effect avoids the drawbacks of combining an “all-in-one” composition, and the drawbacks of layering products not designed to be used together. That is, separating a product in a specific way into two or more layers and applying those layers to skin in a specific sequence, skin feel and skin appearance benefits can be greatly enhanced. The personal care products herein may be used in skin care and cosmetics, non-limiting uses of which include moisturizers, conditioners, anti-aging compounds, skin lightening compounds, and combinations thereof. The composition is applied to keratinous tissue of the face, neck, hands, arms and other areas of the body.

Percentages are by weight of the personal care composition or the particular phase being described, unless otherwise specified. All ratios are weight ratios, unless specifically stated otherwise. All numeric ranges are inclusive of narrower ranges. The number of significant digits conveys neither limitation on the indicated amounts nor on the accuracy of the measurements. All measurements are understood to be made at ambient conditions, where “ambient conditions” means conditions at about 25° C., under about one atmosphere of pressure, and at about 50% relative humidity unless otherwise noted. As used in the description of the invention and the appended claims, the singular forms “a,” “an,” and “the” are intended to include the plural forms as well, unless the context clearly indicates otherwise.

### DEFINITIONS

“Apply” or “application,” as used in reference to a composition, means to apply or spread the composition onto a keratinous tissue surface.

“Derivative” refers to a molecule similar to that of another one, but differing from it in respect of a certain functional moiety. Derivatives may be formed by known reactive pathways. Suitable functional moieties include esters, ethers, amides, amines, carboxylic acids, hydroxyls, halogens, thiols, and/or salt derivatives of the relevant molecule.

“Free of” means that the stated ingredient has not been added to the composition. However, the stated ingredient may incidentally form as a byproduct or a reaction product of the other components of the composition. “Substantially free of” means that less than 3% (e.g., less than 1%, less than 0.5%, less than 0.25%, or even less than 0.1%) by weight of the composition of the stated ingredient has been added to the composition.

“Keratinous tissue” refers to keratin-containing layers disposed as the outermost protective covering of mammals which includes, but is not limited to, skin, hair, nails, cuticles, etc.

“Non-volatile” means a material that exhibit a vapor pressure of no more than about 0.2 mm Hg at 25° C. at one atmosphere and/or a material that has a boiling point at one atmosphere of at least about 300° C. “Volatile” means that the material exhibits a vapor pressure of at least about 0.2 mm of mercury at 25° C.

“Personal care product” or “personal care composition” means a product or composition suitable for topical application on mammalian keratinous tissue.

“Regulating skin condition” means improving skin appearance and/or feel, for example, by providing a benefit, such as a smoother appearance and/or feel. Herein, “improving skin condition” means effecting a visually and/or tactilely perceptible positive change in skin appearance and feel. The benefit may be a chronic or acute benefit and may include one or more of the following: reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

“Signs of skin aging,” include, but are not limited to, all outward visibly and tactilely perceptible manifestations, as well as any macro- or micro-effects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Safe and effective amount” means an amount of a compound or composition sufficient to induce a positive benefit but low enough to avoid serious side effects (i.e., provides a reasonable benefit to risk ratio within the judgment of a skilled artisan).

“Skin” means the outermost protective covering of mammals that is composed of cells such as keratinocytes, fibroblasts and melanocytes. Skin includes an outer epidermal layer and an underlying dermal layer. Skin may also include hair and nails as well as other types of cells commonly associated with skin, such as, for example, myocytes, Merkel cells, Langerhans cells, macrophages, stem cells, sebocytes, nerve cells and adipocytes.

“Skin-care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin-care active” means a compound or combination of compounds that, when applied to skin, provide an acute and/or chronic benefit to skin or a type of cell commonly found therein. Skin-care actives may regulate and/or improve skin or its associated cells (e.g., improve skin elasticity; improve skin hydration; improve skin condition; and improve cell metabolism).

“Skin-care composition” means a composition that includes a skin-care active and regulates and/or improves skin condition.

“Skin-care product” as used herein refers to a product that includes a skin-care composition. Some nonlimiting examples of “skin-care products” include skin creams, moisturizers, lotions, and body washes.

“Topical application” means to apply or spread the compositions of the present invention onto the surface of the keratinous tissue.

“UV agent” means a material or composition recognized by a skilled artisan in the field of sunscreen formulation to be a dermatologic ally acceptable ultraviolet radiation absorbing and/or scattering material. Such UV actives may be described as being UV-A and/or UV-B active agents. Approval by a regulatory agency is generally required for inclusion of UV agents in formulations intended for human use. Exemplary UV agents are described in more detail below.

### Multi-Step Regimen for Providing Look and Feel Benefits to a Target Skin Portion.

Described herein is a method of providing benefits to human skin, specifically, the benefits of simultaneously protecting skin and improving the look and feel of skin. Even more specifically, the first and second layer compositions may be applied to skin exhibiting signs of skin aging, for example, to reduce the appearance of wrinkles, which include reducing the appearance of wrinkles, reducing the appearance of deep lines, reducing the appearance of fine lines, reducing the appearance of large pores and bumps on the skin. Further the skin is hydrated and moisturized. The individual ingredients of the compositions herein, both required and optional, as well as their properties and concentration levels, are defined in greater detail below.

There is provided herein a multi-step process where the first step is applying a first layer, which is a skin care product. The second step is applying a second layer, which is a finisher composition comprising high levels of particulate material to provide a suitable powder layer. In some instances, the skin care product is a moisturizing composition, and may include a relatively high level of humectant. In order to provide the overall desired look and feel benefits from the multi-step regimen, the second layer or powder layer contains non-volatile oils (e.g., silicone oils and/or liquid UV agents) in specific ratios with respect to the particulate concentration.

The layers of the present compositions are kept separate until use to insure they can be applied in the proper order, which, as the comparative examples will illustrate, is an important aspect of the present method. The compositions can, however, be sold in a variety of ways. The multiple layers can be sold in the same package, if separated until dispensed. The compositions can be sold in individual containers that are sold separately or together. Non-limiting examples include one box that contains multiple bottles, each bottle containing a different layer, or the layers can be sold separately in an array of packages where the consumer can select from various different versions of each layer.

The compositions may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices. After application, the composition may be allowed to remain on the skin.

The amount of the composition applied, the frequency of application and the period of use will vary widely depending upon the level of components of a given composition and the level of regulation desired. For example, from about 0.1 mg composition/cm² to about 50 mg composition/cm², and alternatively about 2 mg composition/cm² of skin may be applied. In one embodiment, the composition is applied at least once daily, where “daily” and “days” mean a 24-hour period. The user may be instructed to reapply the composition after a period of time has passed, for example every hour, and alternatively when the composition has been washed or rinsed from the skin, for example after washing one's hands or face, or after swimming, bathing and showering. The compositions may be applied as part of a treatment regimen, for example, once daily for 30 consecutive days, alternatively for 14 consecutive days, alternatively for 7 consecutive days and alternatively for 2 consecutive days.

### Personal-Care Compositions for Use in a Multi-Step Regimen.

The personal-care compositions of the present invention may be skin care or cosmetic products. The personal-care compositions may be used as, for example, a moisturizer, conditioner, anti-aging compound, or skin-lightening compound. In certain embodiments, the composition is applied to the face, neck, hands, arms, and other typically exposed areas of the body.

The compositions herein are useful for improving skin appearance and feel. The compositions of the present invention may be useful for regulating skin condition and improving skin condition. In some instances, the compositions are useful for regulating and improving the signs of skin aging. The compositions may provide an essentially immediate (i.e., acute) improvement in skin appearance and feel following application. It is believed that the acute improvement may be attained with a single or limited number of applications of the composition. However, the compositions may comprise components that provide a gradual (i.e., chronic) improvement in skin appearance and feel. It is believed that the chronic improvement may involve multiple, reoccurring, or periodic applications of the composition. The compositions of the present invention may be incorporated into consumer products. In certain embodiments, the consumer products allow the composition to be applied as a spot treatment over a limited area of the skin. In one embodiment, the compositions of the present invention yield a visibly noticeable reduction in wrinkles or bumps on the skin.

The first layer composition herein (i.e., the composition that is applied to the skin first in the multi-step regimen described in more detail below) can be any suitable skin care composition or product (e.g., moisturizer, conditioner, anti-aging product, skin-lightening product or other skin care product). The first layer compositions may be in the form of an aqueous solution, aqueous gel, aqueous dispersion, oil-in-water emulsion, or a water-in-oil-in-water emulsion.

The second layer composition herein is a powder layer and may be referred to as a “finisher” or “finisher composition.” Finishers are generally recognized in the cosmetics industry as compositions that are applied as a topcoat (i.e., overlying layer) to a basecoat (i.e., underlying layer) of composition such as the skin care composition described above. The finisher herein is an oil-in-water emulsion. The oil phase of the finisher includes a non-volatile oil (e.g., silicone oils such as dimethicone, liquid UV agents, and non-silicone oils such as hydrocarbon oils, esters, ethers, and the like). The powders, oils and other ingredients that may be included in the present finisher are described in more detail below.

The aqueous phase of the first and/or second layers typically includes water. The first layer may comprise 20% to 99% (40% to 90% or even from 50% to 85%) of water. The second layer may comprise 20% to 85% (e.g., 30% to 80% or even from 40% to 75%) of water. The aqueous phase of either the first layer or the powder layer may comprise components other than water (non-water components), including but not limited to water-soluble moisturizing agents, conditioning agents, anti-microbials, humectants and other water-soluble skin care actives, to impart an increased benefit to the keratinous tissue.

Humectant

The first layer herein (i.e., the skin care composition or product) may include one or more humectants at an amount suitable for providing a skin moisturizing and/or other skin care benefit. The first layer of skin care composition may comprise from about 0% to about 30%, alternatively, from about 5% to about 25%, or from about 10% to about 20%, of the humectant, when present. Glycerin is a particularly suitable humectant for use in the skin care composition of the first layer and can be the sole humectant used. In some instances, the humectant may include at least 50% or at least 75%, by weight, of glycerin.

The second layer finisher composition herein may optionally include a humectant. However, the amount of humectant present in the finisher composition should be low enough to enable the finisher to provide the desired look and feel benefit to the underlying skin care product. For example, the finisher composition may optionally include less than 10% or even less than 5% by weight of glycerin. It may be desirable to formulate the finisher composition such that it is free of or substantially free of humectants such as glycerin.

An exemplary class of humectants is polyhydric alcohols. Suitable polyhydric alcohols include polyalkylene glycols and alkylene polyols and their derivatives, including propylene glycol, dipropylene glycol, polypropylene glycol, polyethylene glycol and derivatives thereof; sorbitol; hydroxypropyl sorbitol; erythritol; threitol; pentaerythritol; xylitol; glucitol; mannitol; butylene glycol (e.g., 1,3-butylene glycol); pentylene glycol; hexane triol (e.g., 1,2,6-hexanetriol); glycerin; ethoxylated glycerine; and propoxylated glycerine.

Other suitable humectants include sodium 2-pyrrolidone-5-carboxylate, guanidine; glycolic acid and glycolate salts (e.g., ammonium and quaternary alkyl ammonium); lactic acid and lactate salts (e.g., ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; sodium pyroglutamate, water-soluble glyceryl poly(meth)acrylate lubricants (such as Hispagel®) and mixtures thereof.

Powder System

The finisher composition herein includes a suitable powder system. The powder system provides a light diffusing effect that provides a smooth look to the skin that is often more natural looking than makeup. Alternatively or additionally, the powder system may provide a silky or lubricious feel that can offset the undesirable greasiness associated with oils and/or the undesirable tacky feel associated with some humectants. It is important for the powder system to include suitable level of powder particles. If too much powder is present, then the look and feel benefits provided by the finisher composition herein may level off or even start to decline. In particular, the powder may no longer remain evenly distributed on the skin surface, which can lead to undesirable whitening (e.g., because powders no longer remain wetted) and/or flaking from the skin (e.g., because the powders no longer suitably adhere to the rest of the product film). On the other hand, if too little powder is present in the finisher composition, then the undesirable look and/or feel properties of the underlying skin care composition may not be altered as desired.

It is believed, without being limited by theory, that the size of the particles in the powder is also important for delivering visible texture benefits on skin. In particular, it is important that the particles are large enough to protrude from the dry film formed by the skin care product on the skin (i.e., at least a portion of each (or most) of the particle(s) extends out of the surface of the film). In this way, a “rough” film is created, which diffusely reflects light (i.e., creates a bumpy-looking surface) and reduces the surface area of the underlying skin care product film that can be contacted by a user's hand or other object (i.e., reduces the tacky and/or greasy feel of the skin care composition, etc.). But as particle size increases, the number of particles in the finisher composition decreases. For substantially spherical particles, the number of particles per unit volume is proportional to the inverse of the cube of the particle diameter. Thus, using relatively large particles at a fixed amount (i.e., weight percent) of powder in the product effectively reduces the number of particles that can be added. On the other hand, using smaller particles may increase the number of particles present in the finisher, but may not provide the desired “rough surface” to the product film because a smaller portion of each particle (or even no portion of the particle) extends above the surface of the product film. On average, the dry film thickness of a conventional skin care product, when used as intended, typically ranges between 1 and 6 microns. Consequently, it is important to ensure that the selected particle size is appropriate for the skin care product it is intended to be used with. Particle size can be determined by any suitable method known in the art, such as by using coulter-counter equipment or the ASTM Designation E20-85, titled “Standard Practice for Particle Size Analysis of Particulate Substances in the Range of 0.2 to 75 Micrometers by Optical Microscopy,” ASTM Volume 14.02, 1993. The particle sizes disclosed herein are volume-weighted mean particle sizes.

The particles in the powder systems disclosed herein are substantially spherical (i.e., the majority or even all the particles in the finisher composition are spherical). It is believed, without being limited by theory, that spherical particles generally provide a more suitable product feel relative to non-spherical particles, at least in part because a spherical particle creates less drag and rolls more smoothly across a surface than a non-spherical particle. As used herein, “spherical” and “sphere” mean particles that have an aspect ratio (i.e., ratio of major axis to minor axis) of from 1:1 to 2:1, (e.g., 1:1 to 2:1, 1:1 to 1.6:1 or even 1:1 to 1.4:1). The shape of the particles may be determined by any suitable method known in the art (e.g., optical microscope or electron microscope and suitable image analysis software).

In some instances, the powder system may include spherical silicone elastomer particles. For example, the finisher may include from 10% to 25% by weight of spherical silicone elastomer particles (e.g., from 12% to 25% or even 14% to 20%) dispersed or suspended in a suitable carrier. The amount of silicone elastomer powder in the finisher is determined based on the particulate material being in neat form (i.e., not swollen in solvent). It may be desirable to provide spherical silicone elastomer particles that have no tackiness and a rubber hardness (as measured by Durometer A defined in JIS K 6253) in the range of 10 to 90, (e.g., 20 to 80 or even from 25 to 75). When the rubber hardness is less than 5, the resulting silicone particles tend to become agglomerated, and dispersion into primary particles can be difficult. In contrast, a rubber hardness in excess of 90 may invite loss of soft texture that undesirably affects the feel properties provided by the finisher. The spherical silicone elastomer particles herein may have a median particle size of from 2 μm to 40 μm, (e.g., from 4 μm to 30 μm or even from 5 μm to 15 μm). Of course, it is to be appreciated that the particle sizes disclosed herein may be readily adapted for use with thicker or thinner films without departing from the spirit and scope of the present invention.

Spherical silicone particles suitable for use herein may be prepared from a variety of silicone materials, e.g., organopolysiloxanes such as cured silicone rubbers and poly(organosilsesquioxane) resins. The silicone elastomer particles suitable herein may be coated or uncoated. For example, the silicone particles may include silicone resin-coated silicone rubber particles (e.g., silicone rubber particles with polyorganosilsesquioxane attached to their surface). Commercially available silicone particles suitable for use in the present invention include: KSP-100, -101, -102, -103, -104, and -105, all from Shin Etsu; and DC9506 and DC 9701 from Dow Corning.

In some instances, the present finisher may include from 10% to 25% by weight of spherical starch particles (e.g., from 15% to 25% or even 20%) dispersed or suspended in a suitable carrier. The starch particles suitable for use herein may be coated or uncoated (e.g., coated with a suitable silicone material). In some instances, the starch particles may be a coated or uncoated starch derivative. The starch particles herein may have a median particle size of from 5 μm to 30 μm, (e.g., from 8 μm to 25 μm or even from 10 μm to 20 μm). Some non-limiting examples of commercially available starch particles suitable for use herein are tapioca starch (available as Tapioca Pure from AkzoNobel), corn starch (available as Purity 21C from AkzoNobel), potato starch, glyceryl starch (available as Dry-Flo GS from AkzoNobel), aluminum starch octenylsuccinate (available as Mackaderm ASTO-Dry from Rhodia, Inc., and Dry-Flo PC from AkzoNobel), calcium starch octenylsuccinate (available as Skin Flow C from MGP Ingredients, Inc., and Mackaderm CSTO-Dry from Rhodia, Inc.), and polymethylsilsesquioxane coated tapioca starch (available as Dry-Flo TS from AkzoNobel).

Non-Volatile Oil

In order to improve the skin appearance benefits provided by the finisher and minimize any undesirable visible tradeoffs (e.g., whitening), (i.e., coat, partially coat, or soak) the spherical particles herein are wetted with a non-volatile oil. It is important to ensure that the particles are wetted with the right amount of volatile oil. If the particles are applied to the target skin surface with too little non-volatile oil, the finisher may appear white, and thus any wrinkle or pore masking benefit provided by the finisher composition may be overshadowed by undesirable whiteness. On the other hand, if too much non-volatile oil is present, the skin may appear undesirably shiny, thus reducing or eliminating the skin textural masking benefit of the finisher (i.e., the ability of the finisher to help reduce the appearance of perceived skin flaws related to skin texture, such as wrinkles and pores). Accordingly, it is important to provide a suitable weight ratio of the non-volatile oil to the particles of between 1:10 and 1:1 (e.g., from 1:5 to 4:5 or even from 1:4 to 3:5).

The non-volatile oil present in the finisher composition should remain on this skin for a relatively long period of time after application (e.g., more than 2 hours, 4 hours, or even more than 8 hours) without significant evaporation or absorption into the skin. If the oil evaporates, as a volatile oil would, or is absorbed into the skin, unwetted particles may be left on the skin resulting in undesirable whiteness. In addition, it may be desirable to select a non-volatile oil with a low refractive index, since high refractive index oils tend to make the skin appear shiny, which may reduce or even eliminate the skin textural masking benefit of the finisher.

The liquid UV agent(s) described in more detail below are particularly suitable non-volatile oils for use in the finisher compositions herein. However, in some instances, the oil phase of the finisher may also include other non-volatile oils such as non-volatile silicone oils, hydrocarbon oils, amides, esters, ethers and mixtures of these. Some non-limiting examples of silicone and hydrocarbon non-volatile oils can be found in copending U.S. Ser. Nos. 14/245,230 and 14/245,241.

UV Agent

To provide UV protection, the current composition contains a UV agent. There are many types of UV agents, but one particularly useful form is liquid UV agents. As used herein “liquid UV agent” means one or more UV agent(s) that is(are) liquid at room temperature. Liquid UV agents include UV agents that are generally recognized as being liquids by those skilled in the art of sunscreen formulation. Liquid UV agents also include oil-soluble solid UV agents that are dissolved in a non-volatile oil to form a UV absorbing/diffusing composition that is a liquid at room temperature. Liquid UV agents tend to be oily or oil-based materials, and are also non-volatile, which is important for the longevity of the finisher on the skin. Thus, the liquid UV agent can provide the benefit desired from the non-volatile oil (e.g., particle wetting and longevity) and a UV protection benefit. Accordingly, the liquid UV agent may be used as the non-volatile oil in the finisher composition in whole or in part. The non-volatile oil of the finisher composition herein includes a liquid UV agent at an amount of at least 50% (e.g., at least 60%, 70%, 80%, 90%, 95%, 97%, 99% or even 100%) by weight of the non-volatile oil.

The UV agent(s) present in the composition(s) herein may be added to provide a desired sun protection factor. For example, a finisher composition herein may have a sun protection factor of 5 or more (e.g., 10, 15, 20, 25, 30, 35, 40, 50, 60, 70 80 90 or even up to 100) when used as intended on skin. The SPF of the finisher composition may range from 4 to 100, from 8 to 55, or even from 12 to 35, as desired.

Some nonlimiting examples of known UV agents include Benzophenone, Benzophenone-1, Benzophenone-2, Benzophenone-3 (oxybenzone), Benzophenone-4 (sulisobenzone), Benzophenone-5, Benzophenone-6, Benzophenone-7, Benzophenone-8, Benzophenone-9 (dioxybenzone), Benzophenone-10, Benzophenone-11, Benzophenone-12, Benzotriazolyl Dodecyl p-Cresol, 3-Benzylidene Camphor, Benzylidene Camphor Sulfonic Acid, Benzyl Salicylate, Bis-Ethylhexyloxyphenol Methoxyphenyl Triazine (bemotrizinol), Bornelone, Bumetrizole, Butyl Methoxydibenzoylmethane (avobenzone), Butyl PABA, Cinnamidopropyltrimonium Chloride, Cinoxate, DEA-Methoxycinnamate, Dibenzoxazoyl Naphthalene, Di-t-Butyl Hydroxybenzylidene Camphor, Diethylamino Hydroxybenzoyl Hexyl Benzoate, Diethylhexyl Butamido Triazone (iscotrizinol), Diisopropyl Ethyl Cinnamate, Diisopropyl Methyl Cinnamate, Di-Methoxycinnamidopropyl Ethyldimonium Chloride Ether, Dimethyl PABA Ethyl Cetearyldimonium Tosylate, Dimorpholinopyridazinone, Dimorpholino-pryridazinone, Disodium Bisethylphenyl Triaminotriazine Stilbenedisulfonate, Disodium Distyrylbiphenyl Disulfonate, Disodium Phenyl Dibenzimidazole Tetrasulfonate, Drometrizole, Drometrizole Trisiloxane, Ethyl Dihydroxypropyl PABA, Ethyl Diisopropylcinnamate, Ethylhexyl Bis-Isopentylbenzoxazolylphenyl Melamine, Ethyl Dimethoxybenzylidene Dioxoimidazolidine Propionate, Ethylhexyl Dimethyl PABA, Ethylhexyl Methoxycinnamate (octinoxate), Ethylhexyl Methoxydibenzoylmethane, Ethylhexyl Salicylate (octisalate), Ethylhexyl Triazone (octyl triazone), Ethyl Methoxycinnamate, Ethyl PABA, Ethyl Urocanate, Etocrylene, 4-(2-Beta-Glucopyrano-siloxy) Propoxy-2-Hydroxybenzophenone, Glyceryl Ethylhexanoate Dimethoxycinnamate, Glyceryl PABA, Glycol Salicylate, Hexanediol Disalicylate, Homosalate, Isoamyl Cinnamate, Isoamyl p-Methoxycinnamate, Isopentyl Trimethoxycinnamate Trisiloxane, Isopropylbenzyl Salicylate, Isopropyl Dibenzoylmethane, Isopropyl Methoxy-cinnamate, Kaempferia Galanga Root Extract, Menthyl Anthranilate (meradimate), Menthyl Salicylate, Methoxycinnamidopropyl Hydroxysultaine, Methoxycinnamidopropyl Laurdimonium Tosylate, 4-Methylbenzylidene Camphor (enacamene), Methylene Bis-Benzotriazolyl Tetramethylbutylphenol (bisoctrizole), Octocrylene, Octrizole, PABA, PEG-25 PABA, Phenylbenzimidazole Sulfonic Acid (ensulizole), Polyacrylamidomethyl Benzylidene Camphor, Polyamide-2, Polyquaternium-59, Polysilicone-15 (diethylbenzylidene malonate dimethicone), Potassium Methoxy-cinnamate, Potassium Phenylbenzimidazole Sulfonate, Red Petrolatum, Sodium Benzotriazoyl Butylphenol Sulfonate, Sodium Phenylbenzimidazole Sulfonate, Sodium Urocanate, TEA-Phenylbenzimidazole Sulfonate, TEA-Salicylate, Terephthalylidene Dicamphor Sulfonic Acid (ecamsule), Tetrabutyl Phenyl Hydroxybenzoate, Titanium Dioxide, Urocanic Acid, Zinc Cerium Oxide, Zinc Oxide

Some particularly suitable examples of UV agents that are generally recognized as being liquids are Ethylhexyl Dimethyl PABA, Ethylhexyl Methoxycinnamate (octinoxate), Ethylhexyl Salicylate (octisalate), Homosalate, Isoamyl p-Methoxycinnamate (amiloxate), Menthyl Anthranilate (meradimate), Octocrylene, Polysilicone-15 (diethylbenzylidene malonate dimethicone) and combinations of these.

Some particularly suitable examples of oil soluble solid UV agents, which can be dissolved in a non-volatile oil such as one of the UV agents described above to form a liquid UV agent, are Benzophenone-3 (oxybenzone), Benzophenone-9 (dioxybenzone), Bis-Ethylhexyloxyphenol Methoxyphenyl Triazine (bemotrizinol), Butyl Methoxydibenzoylmethane (avobenzone), Diethylamino Hydroxybenzoyl Hexyl Benzoate, Diethylhexyl Butamido Triazone (iscotrizinol), Drometrizole Trisiloxane, Ethylhexyl Triazone (octyl triazone), 4-Methylbenzylidene Camphor (enacamene) and combinations of these.

### Optional Ingredients

Particles

In some instances, the first layer skin care composition and/or the second layer finisher composition may, optionally, include other particles in addition to the spherical silicone elastomer and/or spherical starch particles herein. For example, the finisher composition may, optionally, include non-spherical particles (e.g., non-spherical silicone elastomer particles, mica, talc, clay). However, in this example, the finisher will include less than 4% of non-spherical particles (e.g., less than 3% or even less than 1%). When referring to non-spherical silicone elastomer particles, the indicated percentages are understood to refer to amount of dry elastomer, as opposed to the total amount of elastomer and solvent, used for example for storage and shipping. Exemplary non-spherical crosslinked siloxane elastomers include the CTFA (Cosmetic, Toiletry, and Fragrance Association International Cosmetic Ingredient Dictionary and Handbook, 11^{th} ed.) designated dimethicone/vinyl dimethicone crosspolymers supplied by a variety of suppliers including Dow Corning™, General Electric™, Shin Etsu™ (KSG 15 and 16), and Grant Industries. Other exemplary non-emulsifying crosslinked siloxane elastomers include the CTFA designated dimethicone crosspolymers including Dow Corning™; e.g. DC 9040 and DC 9045 which are supplied as a 12.5% elastomers in cyclomethicone, and DC 9041 which is supplied as 16% elastomer in dimethicone.

Pigment

It may particularly desirable to keep the amount of pigment present in the finisher composition relatively low, in order to avoid the undesirable aesthetics associated with higher levels of pigment (e.g., whiteness, flaking and lower spreadability). The second layer finisher compositions herein include less than 1%, (e.g., less than 0.5% or even less than 0.1%) by weight of particles that impart chroma and/or opacity to the composition (e.g., pigment grade titanium dioxide or iron oxide). In some instances, the present finisher is free of pigment and/or other colorants (e.g., lakes and dyes). Exemplary pigments can be found the Personal Care Product Council's International Cosmetic Ingredient Dictionary and Handbook, Thirteenth Edition, 2010.

Conventional Ingredients

The first and second layer compositions herein may include one or more optional materials that are commonly used in personal care compositions, such as volatile oils, emulsifiers, thickeners, skin care actives, combinations of these and the like. In particular, it may be desirable in some instances to include a volatile oil in the finisher composition at up to 20 wt % (e.g., from 5% to 20%). The volatile oil may be a volatile silicone, a volatile hydrocarbon oil or a combination of these.

Volatile silicones include cyclic and linear volatile silicones. A description of various volatile silicones is found in Todd, et al. “Volatile Silicone Fluids for Cosmetics”, 91 Cosmetics and Toiletries 27-32 (1976). Suitable cyclic volatile silicones include cyclic dimethyl siloxane chains containing an average of from about 3 to about 5 silicon atoms, preferably from about 4 to about 5 silicon atoms. Exemplary cyclic volatile silicones of varying viscosities include Dow Corning DC 244, DC 245, DC 344, and DC 345; GE Silicones-OSi Specialties Volatile Silicone 7207 and Volatile Silicone 7158; and GE Silicones SF1202. Suitable volatile linear silicones include the polydimethylsiloxanes containing an average of from about 2 to about 8 silicon atoms. Exemplary linear volatile silicones include the Dow Corning DC 200 series with viscosities of 0.65 cst, 1.0 cst, and 2.0 cst. In certain embodiments, the linear volatile silicones generally have viscosities of less than or equal to about 4 centistokes at 25° C., and the cyclic materials generally have viscosities of less than about 6 centistokes at 25° C.

Some non-limiting examples of suitable volatile hydrocarbon oils include isododecane (e.g., Permethyl-99A which is available from Presperse Inc.), isodecane, and the C7-C8 through C12-C15 isoparaffins (e.g., Isopar Series available from Exxon Chemicals).

Emulsifier

The compositions may optionally include an emulsifier. An emulsifier is particularly suitable when the composition is in the form of an emulsion or if immiscible materials are being combined. The compositions may comprise from about 0.05%, 0.1%, 0.2%, 0.3%, 0.5%, or 1% to about 20%, 10%, 5%, 3%, 2%, or 1% emulsifier. Emulsifiers may be nonionic, anionic or cationic. Non-limiting examples of emulsifiers are disclosed in U.S. Pat. No. 3,755,560, U.S. Pat. No. 4,421,769, and McCutcheon's, Emulsifiers and Detergents, 2010 Annual Ed., published by M. C. Publishing Co. Other suitable emulsifiers are further described in the Personal Care Product Council's International Cosmetic Ingredient Dictionary and Handbook, Thirteenth Edition, 2006, under the functional category of “Surfactants—Emulsifying Agents.”

Suitable emulsifiers include the following classes of ethers and esters: ethers of polyglycols and of fatty alcohols, esters of polyglycols and of fatty acids, ethers of polyglycols and of fatty alcohols which are glycosylated, esters of polyglycols and of fatty acids which are glycosylated, ethers of C₁₂₋₃₀ alcohols and of glycerol or of polyglycerol, esters of C₁₂₋₃₀ fatty acids and of glycerol or of polyglycerol, ethers of oxyalkylene-modified C₁₂₋₃₀ alcohols and of glycerol or polyglycerol, ethers of C₁₂₋₃₀ fatty alcohols comprising and of sucrose or of glucose, esters of sucrose and of C₁₂₋₃₀ fatty acids, esters of pentaerythritol and of C₁₂₋₃₀ fatty acids, esters of sorbitol and/or of sorbitan and of C₁₂₋₃₀ fatty acids, ethers of sorbitol and/or of sorbitan and of alkoxylated sorbitan, ethers of polyglycols and of cholesterol, esters of C₁₂₋₃₀ fatty acids and of alkoxylated ethers of sorbitol and/or sorbitan, and combinations thereof. Silicone emulsifiers may be use in the present compositions. Linear or branched type silicone emulsifiers may also be used. Particularly useful silicone emulsifiers include polyether modified silicones such as KF-6011, KF-6012, KF-6013, KF-6015, KF-6015, KF-6017, KF-6043, KF-6028, and KF-6038 and polyglycerolated linear or branched siloxane emulsifiers such as KF-6100, KF-6104, and KF-6105; all from Shin Etsu.

Thickening Agent

The composition of the present invention may include one or more thickening agents. The composition of the present invention may comprise from about 0.1% to about 5%, or, alternatively, from about 0.3% to about 3%, of a thickening agent when present. Suitable classes of thickening agents include but are not limited to carboxylic acid polymers, polyacrylamide polymers, sulfonated polymers, copolymers thereof, hydrophobically modified derivatives thereof, and mixtures thereof.

Suitable thickening agents include carboxylic acid polymers such as the carbomers (e.g., the CARBOPOL® 900 series such as CARBOPOL® 954), and Ultrez 10 and Ultrez 30. Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, Ultrez 20, Ultrez 21, PEMULEN TR-1, and PEMULEN TR-2, from Noveon, Inc.

Other suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.).

Other suitable thickening agents useful herein are sulfonated polymers such as the CTFA designated sodium polyacryloyldimethyl taurate available under the trade name Simulgel 800 from Seppic Corp. and Viscolam At 100 P available from Lamberti S.p.A. (Gallarate, Italy). Another commercially available material comprising a sulfonated polymer is Sepiplus 400 available from Seppic Corp.

Further, suitable thickeners may include superabsorbent polymers. These superabsorbent polymers may be chosen from: crosslinked sodium polyacrylates, such as, for example, those sold under the names Octacare X100, X110 and RM100 by Avecia, those sold under the names Flocare GB300 and Flosorb 500 by SNF, those sold under the names Luquasorb 1003, Luquasorb 1010, Luquasorb 1280 and Luquasorb 1100 by BASF, those sold under the names Water Lock G400 and G430 (INCI name: Acrylamide/Sodium Acrylate Copolymer) by Grain Processing, or Aqua Keep 10 SH NF, Aqua Keep 10 SH NFC, sodium acrylate crosspolymer-2, provided by Sumitomo Seika, starches grafted by an acrylic polymer (homopolymer or copolymer) and in particular by sodium polyacrylate, such as those sold under the names Sanfresh ST-100C, ST100MC and IM-300MC by Sanyo Chemical Industries (INCI name: Sodium Polyacrylate Starch), hydrolysed starches grafted by an acrylic polymer (homopolymer or copolymer), in particular the acryloacrylamide/sodium acrylate copolymer, such as those sold under the names Water Lock A-240, A-180, B-204, D-223, A-100, C-200 and D-223 by Grain Processing (INCI name: Starch/Acrylamide/Sodium Acrylate Copolymer). Preferred superabsorbent polymers include Makimousse 12 and Makimouse 25 supplied by Kobo Products Inc.

Suitable thickeners for use herein include gums. “Gum” is a broadly defined term in the art. Gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, derivatives thereof and mixtures thereof.

Natural gums are polysaccharides of natural origin, capable of causing a large viscosity increase in solution, even at small concentrations. They can be used as thickening agents, gelling agents, emulsifying agents, and stabilizers. Most often these gums are found in the woody elements of plants or in seed coatings. Natural gums can be classified according to their origin. They can also be classified as uncharged or ionic polymers (polyelectrolytes), examples of which include the following. Natural gums obtained from seaweeds, such as: agar; alginic acid; sodium alginate; and carrageenan. Natural gums obtained from non-marine botanical resources include: gum arabic, from the sap of Acacia trees; gum ghatti, from the sap of Anogeissus trees; gum tragacanth, from the sap of Astragalus shrubs; karaya gum, from the sap of Sterculia trees. Examples of uncharged gums include: guar gum, from guar beans, locust bean gum, from the seeds of the carob tree; beta-glucan, from oat or barley bran; chicle gum, an older base for chewing gum obtained from the chicle tree; dammar gum, from the sap of Dipterocarpaceae trees; glucomannan from the konjac plant; mastic gum, a chewing gum from ancient Greece obtained from the mastic tree; psyllium seed husks, from the Plantago plant; spruce gum, a chewing gum of American Indians obtained from spruce trees; tara gum, from the seeds of the tara tree. Natural gums produced by bacterial fermentation include gellan gum and xanthan gum.

Actives

The composition of the present invention may comprise at least one skin care active (“active”), useful for regulating and/or improving the condition of mammalian skin. The active may be soluble in oil or water, and may be present primarily in the oil phase and/or in the aqueous phase. Solubility in water and oil is within the knowledge of one of skill in the art, and can be determined using known methods of analysis. One of skill in the art further will understand that solubility may be affected by the type and concentration of other components in the composition, and other conditions such as pH, ionic strength, etc. Many skin care actives may provide more than one benefit, or operate via more than one mode of action; therefore, classifications herein are made for the sake of convenience and are not intended to limit the active to that particular application or applications listed. Suitable actives include, but are not limited to, vitamins, peptides, sugar amines, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, and antifungals.

The compositions of the present invention may comprise from about 0.001% to about 10%, alternatively from about 0.01% to about 5%, of at least one vitamin. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®). The compositions may comprise from about 1×10⁻⁷% to about 20%, alternatively from about 1×10⁻⁶% to about 10%, and alternatively from about 1×10⁻⁵% to about 5% of the peptide.

### Methods

### Chroma

Herein, “chroma” describes color and color intensity. For the purposes of the present invention, color is defined according to a value on the CIELAB color system, which is based on the XYZ color system, defined by the Commission Internationale de l'Eclairage to provide a manner of objectively representing perceived color and color differences. X, Y and Z can be expressed in a variety of manners, or “scales,” one of which is the Hunter scale. The Hunter scale has three variables, L, a, and b, which correlate mathematically to X, Y and Z, and is described by Robertson, A. R. in “The CIE 1976 Color Difference Formulas,” Color Research Applications, vol. 2, pp. 7-11 (1977).

To measure the color of the compositions of the present invention, a thick, uniform film of the composition is first created on a standard background. Specifically, product is applied onto a standard opacity chart such as Form N2A commercially available from the Leneta Company of Manwah, N.J. or equivalent, of which the top half is black and the bottom half is white, and then spread on the black area of the opacity chart into a film having a thickness of approximately 250 microns using a film applicator (e.g., as commercially available from BYK Gardner of Columbia, Md., or the equivalent thereof).

The color (L, a, and b values) of the product film is then measured using a spectrophotometer with settings selected to exclude specular reflection. The value for “a” correlates to a value along the red-green (horizontal) axis, and the value for “b” correlates to a value along the blue-yellow (vertical) axis. For example, a blue-colored sample will have a negative b-value, whereas a red-colored sample will have a positive a-value. A more positive or negative value represents a more intense color. The value for “L” is an indicator of lightness and/or darkness, and correlates to a value along the z-axis, which is perpendicular to both the horizontal and vertical axes.

Chroma is measured by a vector having its origin at the intersection of the red-green and blue-yellow axes and extending outward into the color space defined by the horizontal and vertical axes of the CIELAB color system. The length of the vector represents the chroma, and the direction of the vector represents the hue. The shorter the vector, the less colored is the composition, and the lower the chroma. The chroma for the individual layers of the present invention, that is the chroma value for the first layer or the powder layer is less than about 10, preferably less than about 6, and even more preferably less than about 3.

### Contrast Ratio

Herein, “contrast ratio” refers to the opacity of the composition (i.e., the ability of the composition to reduce or prevent light transmission), determined after the composition is drawn onto an opacity chart (Form N2A, Leneta Company of Manwah, N.J. or the equivalent thereof) Contrast Ratio is measured using a spectrophotometer with settings selected to exclude specular reflection. The composition is applied to the top of the opacity chart and then is drawn into a film having a thickness of approximately 38 microns using a film applicator (e.g., as commercially available from BYK Gardner of Columbia, Md., or the equivalent thereof). The film is allowed to dry for 2 hours under conditions of 22° C.+/−1° C., 1 atm. Using a spectrophotometer with the settings selected to exclude specular reflection, the Y tristimulus value (i.e., the XYZ color space of the film) of the product film is measured and recorded. The Y tristimulus value is measured in three different areas of the product film over the black section of the opacity chart, and also in three different areas of the product film over the white section of the opacity chart.

The contrast ratio for the individual layers herein, (e.g., the first layer or the powder layer) is less than about 20, preferably less than about 10, and even more preferably less than about 6.

The contrast ratio is calculated as the mathematical average of the three Y tristimulus values over the black areas, divided by the mathematical average of the three Y tristimulus values over the white areas, times 100:

$$Contrast Ratio = \frac{average ( Yblack )}{average ( Ywhite )} \times 100$$

Visual Attribute Test (VAT)

The visible attribute test (VAT) is a technical panel used to quantify visible benefits of the compositions of the present invention when applied to facial skin. Fifteen to thirty female panelists who are pre-screened to have moderate or higher baseline levels of facial attributes such as fine lines, wrinkles, bumpy surface texture, and pores participate in each VAT study. Two trained expert graders then grade various attributes on each panelist's face both at baseline and 10 minutes after application of 0.45 grams of product to one side of the face. Reductions in facial attributes are then calculated as pre-treatment grade minus the post-treatment grade, and the significance of the differences are determined using ANOVA procedures (Tukey's LSD test).

Table 1 is a hypothetical data table representing typical VAT data calculations for bumpy surface texture.

TABLE 1

| | | | Delta |
|---|---|---|---|
| Panelist | Pre-Treatment | Post-Treatment | (Pre Minus |
| Number | Grade | Grade | Post) |
| 1 | 3.65 | 3.15 | 0.5 |
| 2 | 3.5 | 2.95 | 0.55 |
| 3 | 4.1 | 3.2 | 0.9 |
| 4 | 4.5 | 3.85 | 0.65 |
| 5 | 3.7 | 2.8 | 0.9 |

The facial attributes evaluated by the expert graders include the following:

Bumpy Surface—Skin unevenness or roughness associated with a “pebbled” or an “orange peel” surface. Based on both the degree of roughness as defined as height and proximity and the percentage of the face covered by the surfaced appearance. Roughness and coverage are equally weighted in the final grade. The bumpy surface score should not include obviously raised moles.

Shine—Light reflection on the skin surface. This should include both natural and artificial (product driven and characterized by an oily, greasy look) appearance. The expert graders rate each of the above attributes both pre- and post-treatment using the 5-point continuous line scale shown below:

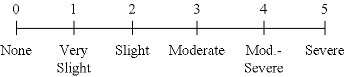

### EXAMPLES

### Examples 1-10

Tables 2 and 3 show the ingredients used to make the first layer compositions of Examples 1 to 10, which are suitable for use as underlying skin composition layer. The first layer compositions of Examples 1 to 10 are prepared by first combining the water phase ingredients and mixing until uniform, warming if necessary. Next, the thickeners are added and the composition is again mixed until uniform. Finally, the pH adjustor, if present, is added and composition is mixed until uniform.

TABLE 2

| | Example 1 | Example 2 | Example 3 | Example 4 | Example 5 |
|---|---|---|---|---|---|
| | First Layer | First Layer | First Layer | First Layer | First Layer |
| Water Phase: | | | | | |
| Water | Qs | qs | qs | qs | Qs |
| Glycerin | 15.0 | 15.0 | 15.0 | 25.0 | 25.0 |
| Dipropylene Glycol | — | — | — | — | 5.0 |
| Butylene Glycol | — | — | 3.0 | — | — |
| Disodium EDTA | 0.05 | 0.05 | 0.05 | 0.05 | 0.05 |
| Symdiol 68¹ | 0.7 | 0.7 | 0.7 | 0.7 | 0.7 |
| Glycacil L² | 0.09 | 0.09 | 0.09 | 0.09 | 0.09 |
| Phenoxyethanol | — | — | — | 0.5 | — |
| Niacinamide | 5.0 | 5.0 | 2.0 | 3.5 | 1.0 |
| D-panthenol | 0.5 | 1.0 | 0.25 | 0.5 | — |
| Sepiwhite MSH³ | 0.2 | 0.5 | — | — | — |
| Glyco-Repair⁴ | 3.0 | — | 1.0 | — | 2.0 |
| Biomyox⁵ | 2.0 | 0.5 | — | — | 1.0 |
| Palmitoyl-pentapeptide⁶ | 0.01 | — | — | — | — |
| N-acetyl glucosamine | — | — | — | 2.0 | 0.5 |
| Inositol | 1.0 | 0.5 | — | — | — |
| Olivem 460⁷ | — | — | 0.1 | — | — |
| Aloe Vera Gel | — | 0.2 | — | — | 0.5 |
| Green Tea Extract | 0.5 | — | — | 1.0 | — |
| pH Adjustor: | | | | | |
| Triethanolamine | 0.1 | 0.3 | — | — | 0.2 |
| Thickener: | | | | | |
| Sepigel 305⁸ | 2.0 | — | — | — | — |
| Simulgel INS-100⁹ | — | 2.5 | 1.5 | — | 1.0 |
| Makimousse-12¹⁰ | — | — | — | 0.6 | — |
| Ultrez 10¹¹ | — | — | — | — | 0.2 |
| Total: | 100% | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³Undecylenoyl phenylalanine, from Seppic
⁴Water and hydrolyzed ceratonia siliqua seed extract, from Silab
⁵Water and nasturtrium officinale extract, from Silab
⁶Palmitoyl-lysine-threonine-threonine-lysine-serine available from Sederma (France)
⁷Sodium PEG-7 olive oil carboxylate, from B&T S.r.l.
⁸Polyacrylamide, C13-14 isoparaffin, and laureth-7, from Seppic
⁹Sodium acrylate/sodium acryloyldimethyl taurate copolymer, isohexadecane, and polysorbate 60, from Seppic
¹⁰Sodium polyacrylate starch, from Kobo Products Inc.
¹¹Carbomer, from Lubrizol

TABLE 3

| | Example 6 | Example 7 | Example 8 | Example 9 | Example 10 |
|---|---|---|---|---|---|
| | First Layer | First Layer | First Layer | First Layer | First Layer |
| Water Phase: | | | | | |
| Water | Qs | qs | qs | qs | Qs |
| Glycerin | 15.0 | 15.0 | 10.0 | 10.0 | 10.0 |
| Propylene Glycol | 3.0 | 5.0 | — | 5.0 | 5.0 |
| Dipropylene Glycol | 3.0 | — | 10.0 | 5.0 | 2.0 |
| Butylene Glycol | 3.0 | — | — | 5.0 | — |
| Disodium EDTA | 0.05 | 0.05 | 0.05 | 0.05 | 0.05 |
| Methylparaben | 0.1 | — | — | — | — |
| Symdiol 68¹ | — | 0.7 | 0.7 | 0.7 | — |
| Sodium Benzoate | 0.1 | — | — | — | — |
| Glycacil L² | — | 0.09 | 0.09 | 0.09 | — |
| Phenoxyethanol | 0.5 | 0.1 | — | — | — |
| Glydant Plus Liquid³ | — | — | — | — | 0.3 |
| Niacinamide | 3.0 | 2.0 | — | — | — |
| D-panthenol | 0.7 | 0.3 | 0.5 | — | — |
| Glyco-Repair⁴ | — | — | — | — | 1.0 |
| Biomyox⁵ | — | 1.5 | — | — | — |
| Palmitoyl-pentapeptide⁶ | 0.03 | — | — | — | 0.01 |
| N-acetyl glucosamine | — | — | 1.0 | — | — |
| Aloe Vera Gel | — | — | — | — | 0.1 |
| Green Tea Extract | — | 0.5 | — | — | 0.1 |
| pH Adjustor: | | | | | |
| Triethanolamine | — | 0.3 | — | — | — |
| Aminomethyl propanol | — | — | — | 0.1 | 0.1 |
| Thickener: | | | | | |
| Simulgel INS-100⁷ | 2.5 | — | — | 0.5 | — |
| Makimousse-7⁸ | — | — | — | — | 0.6 |
| Makimousse-12⁹ | — | — | 0.4 | 0.3 | — |
| Ultrez 10¹⁰ | — | — | — | 0.1 | — |
| Ultrez 21¹¹ | — | 0.3 | — | — | — |
| Xanthan gum | — | — | 0.1 | — | — |
| Total: | 100% | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³DMDM Hydantoin, Butane-1,3-diol, iodopropynyl butylcarbamate, water, from Lonza
⁴Water and hydrolyzed ceratonia siliqua seed extract, from Silab
⁵Water and nasturtrium officinale extract, from Silab
⁶Palmitoyl-lysine-threonine-threonine-lysine-serine available from Sederma (France)
⁷Sodium acrylate/sodium acryloyldimethyl taurate copolymer, isohexadecane, and polysorbate 60, from Seppic
⁸Sodium polyacrylate starch, from Kobo Products Inc.
⁹Sodium polyacrylate starch, from Kobo Products Inc.
¹⁰Carbomer, from Lubrizol
¹¹Acrylates C10-/30 alkyl acrylate crosspolymer, from Lubrizol

### Examples 11-17

Examples 11-17 shown in Table 4 illustrate finisher compositions suitable for use herein. The compositions illustrated in Example 11-17 include a silicone elastomer powder system. The compositions in these examples are prepared in the following manner. In a suitable vessel, the water phase ingredients are combined and heated to 75° C. In a separate suitable vessel, the oil phase ingredients are combined and heated to 75° C. Next the oil phase is added to the water phase and the resulting emulsion is milled (e.g., with a rotor-stator mill The thickener is then added to the emulsion and the emulsion is cooled to 45° C. while stirring. At 45° C., the remaining additional ingredients are added. The product is then cooled with stirring to 30° C., milled again, and then poured into suitable containers.

TABLE 4

| | Ex. 11 | Ex. 12 | Ex. 13 | Ex. 14 | Ex. 15 | Ex. 16 | Ex. 17 |
|---|---|---|---|---|---|---|---|
| Water Phase: | | | | | | | |
| Water | qs | qs | qs | qs | qs | qs | qs |
| Disodium EDTA | 0.1 | 0.05 | 0.05 | 0.1 | 0.05 | 0.05 | 0.05 |
| Benzyl Alcohol | 0.5 | 0.5 | — | 0.5 | — | — | — |
| Methylparaben | 0.25 | 0.25 | — | 0.2 | — | — | — |
| Iodopropynyl Butylcarbamate | 0.09 | 0.09 | 0.09 | 0.09 | 0.09 | 0.09 | 0.09 |
| Phenylbenzimidazole Sulfonic Acid | 1.0 | — | — | 2.0 | — | — | — |
| Symdiol 68¹ | — | — | 0.7 | — | 0.7 | 0.7 | 0.7 |
| Phenoxyethanol | — | — | 0.5 | — | 0.5 | 0.5 | 0.5 |
| Oil Phase: | | | | | | | |
| Isopropyl Isostearate | 2.0 | 0.5 | — | 2.0 | — | — | — |
| Isopropyl lauroylsarcosinate | — | — | — | 3.0 | — | — | — |
| Octisalate | 4.0 | 4.5 | — | — | — | — | — |
| Homosalate | — | 4.0 | — | 8.0 | — | — | — |
| Octocrylene | 1.0 | 2.6 | — | 2.25 | — | — | — |
| Octinoxate | — | — | 4.0 | — | 4.0 | 7.5 | 4.0 |
| Avobenzone | 2.0 | 3.0 | 2.0 | 3.0 | 2.0 | 3.0 | 2.0 |
| Solastay S1² | — | — | 0.5 | — | — | 1.0 | — |
| Stearic Acid | 0.1 | 0.1 | 0.05 | 0.05 | 0.05 | 0.05 | — |
| Ethylparaben | 0.2 | 0.2 | — | 0.2 | — | — | — |
| Propylparaben | 0.15 | 0.15 | — | 0.15 | — | — | — |
| Cetyl alcohol | 0.5 | 0.5 | 0.3 | 0.4 | 0.3 | 0.3 | 0.3 |
| Stearyl alcohol | 0.5 | 0.5 | 0.4 | 0.4 | 0.4 | 0.4 | 0.4 |
| Behenyl alcohol | 0.5 | 0.5 | 0.4 | 0.3 | 0.4 | 0.4 | 0.4 |
| Cetearyl Glucoside | 0.4 | 0.4 | 0.2 | 0.2 | 0.2 | 0.2 | 0.2 |
| PEG-100 stearate | 0.2 | 0.3 | 0.2 | 0.2 | 0.2 | 0.2 | 0.2 |
| Thickener: | | | | | | | |
| Xanthan Gum | — | — | — | — | — | 0.1 | 0.1 |
| Carbopol Ultrez 10³ | 0.2 | — | — | 0.2 | — | — | — |
| Simulgel INS-100⁴ | — | 1.0 | — | — | — | — | — |
| Sepigel 305⁵ | 1.0 | — | — | — | — | — | — |
| Makimousse-12⁶ | — | — | 0.4 | — | 0.4 | 0.4 | 0.4 |
| Simulgel EG⁷ | — | — | — | 2.25 | — | — | — |
| Powders: | | | | | | | |
| KSP 100⁸ | — | 10.0 | — | 5.0 | 8.0 | — | 5.0 |
| KSP 101⁹ | — | 5.0 | — | 5.0 | — | — | — |
| KSP 102¹⁰ | 10.0 | — | — | — | — | 8.0 | — |
| KSP 103¹¹ | — | — | — | 5.0 | — | — | 3.0 |
| KSP 105¹² | — | — | 5.0 | 10.0 | — | 7.0 | 2.0 |
| DC 9506¹³ | — | — | 5.0 | — | 3.0 | — | — |
| Total: | 100% | 100% | 100% | 100% | 100% | 100% | 100% |

¹1,2-hexanediol and caprylyl glycol, from Symrise
²Ethylhexyl Methoxycrylene, from Hallstar
³Carbomer, from Lubrizol
⁴Sodium acrylate/sodium acryloyldimethyl taurate copolymer, isohexadecane, and polysorbate 80, from Seppic
⁵Polyacrylamide, C13-14 isoparaffin, and laureth-7, from Seppic
⁶Sodium polyacrylate starch, from Kobo Products Inc.
⁷Sodium Acrylate/Sodium Acryloyldimethyl Taurate Copolymer & Isohexadecane & Polysorbate 80, from Seppic
⁸Vinyl dimethicone/methicone silsesquioxane crosspolymer, from Shin Etsu
⁹Vinyl dimethicone/methicone silsesquioxane crosspolymer, from Shin Etsu
¹⁰Vinyl dimethicone/methicone silsesquioxane crosspolymer, from Shin Etsu
¹¹Vinyl dimethicone/methicone silsesquioxane crosspolymer, from Shin Etsu
¹²Vinyl dimethicone/methicone silsesquioxane crosspolymer, from Shin Etsu
¹³Dimethicone/vinyldimethicone crosspolymer, from Dow Corning

### Examples 18-24

The compositions used in Examples 18 to 24 are generally prepared in the same manner used for the compositions of Examples 11-17. Table 5 shows the ingredients used to make Examples 18-24, which utilize a starch powder system.

TABLE 5

| | Ex. 18 | Ex. 19 | Ex. 20 | Ex. 21 | Ex. 22 | Ex. 23 | Ex. 24 |
|---|---|---|---|---|---|---|---|
| Water Phase: | | | | | | | |
| Water | qs | qs | qs | qs | qs | qs | qs |
| Disodium EDTA | 0.1 | 0.05 | 0.05 | 0.1 | 0.05 | 0.05 | 0.05 |
| Benzyl Alcohol | 0.5 | 0.5 | — | 0.5 | — | — | — |
| Methylparaben | 0.25 | 0.25 | — | 0.2 | — | — | — |
| Iodopropynyl Butylcarbamate | 0.09 | 0.09 | 0.09 | 0.09 | 0.09 | 0.09 | 0.09 |
| Phenylbenzimidazole Sulfonic Acid | 1.0 | — | — | 2.0 | — | — | — |
| Symdiol 68¹ | — | — | 0.7 | — | 0.7 | 0.7 | 0.7 |
| Phenoxyethanol | — | — | 0.5 | — | 0.5 | 0.5 | 0.5 |
| Oil Phase: | | | | | | | |
| Isopropyl Isostearate | 0.5 | 0.5 | — | 2.0 | — | — | — |
| Isopropyl lauroylsarcosinate | — | — | — | 3.0 | — | — | — |
| Octisalate | 4.0 | 4.5 | — | — | — | — | — |
| Homosalate | — | 4.0 | — | 8.0 | — | — | — |
| Octocrylene | 1.0 | 2.6 | — | 2.25 | — | — | — |
| Octinoxate | — | — | 4.0 | — | 4.0 | 7.5 | 4.0 |
| Avobenzone | 2.0 | 3.0 | 2.0 | 3.0 | 2.0 | 3.0 | 2.0 |
| Solastay S1² | — | — | 0.5 | — | — | 1.0 | — |
| Stearic Acid | 0.1 | 0.1 | 0.05 | 0.05 | 0.05 | 0.05 | — |
| Ethylparaben | 0.2 | 0.2 | — | 0.2 | — | — | — |
| Propylparaben | 0.15 | 0.15 | — | 0.15 | — | — | — |
| Cetyl alcohol | 0.5 | 0.5 | 0.3 | 0.4 | 0.3 | 0.3 | 0.3 |
| Stearyl alcohol | 0.5 | 0.5 | 0.4 | 0.4 | 0.4 | 0.4 | 0.4 |
| Behenyl alcohol | 0.5 | 0.5 | 0.4 | 0.3 | 0.4 | 0.4 | 0.4 |
| Cetearyl Glucoside | 0.4 | 0.4 | 0.2 | 0.2 | 0.2 | 0.2 | 0.2 |
| PEG-100 stearate | 0.2 | 0.3 | 0.2 | 0.2 | 0.2 | 0.2 | 0.2 |
| Thickener: | | | | | | | |
| Xanthan Gum | — | — | — | — | 0.1 | 0.2 | — |
| Carbopol Ultrez 10³ | 0.2 | — | — | 0.2 | — | — | — |
| Simulgel INS-100⁴ | — | 1.0 | — | — | — | — | — |
| Sepigel 305⁵ | 1.0 | — | — | — | — | — | — |
| Makimousse-12⁶ | — | — | 0.4 | — | 0.4 | 0.4 | 0.4 |
| Simulgel EG⁷ | — | — | — | 2.25 | — | — | -- |
| Powders: | | | | | | | |
| Dry Flo TS⁸ | 15.0 | — | 10.0 | 20.0 | — | 10.0 | 9.0 |
| Tapioca Pure⁹ | — | 15.0 | — | 5.0 | 2.0 | 5.0 | 6.0 |
| Dry Flo Pure¹⁰ | — | — | 5.0 | — | 8.0 | 5.0 | — |
| Total: | 100% | 100% | 100% | 100% | 100% | 100% | 100% |

¹1,2-hexanediol and caprylyl glycol, from Symrise
²Ethylhexyl Methoxycrylene, from Hallstar
³Carbomer, from Lubrizol
⁴Sodium acrylate/sodium acryloyldimethyl taurate copolymer, isohexadecane, and polysorbate 80, from Seppic
⁵Polyacrylamide, C13-14 isoparaffin, and laureth-7, from Seppic
⁶Sodium polyacrylate starch, from Kobo Products Inc.
⁷Sodium Acrylate/Sodium Acryloyldimethyl Taurate Copolymer & Isohexadecane & Polysorbate 80, from Seppic
⁸Tapioca and polymethylsilsesquioxane, from Akzo Nobel
⁹Tapioca powder, from Akzo Nobel
¹⁰Aluminum Starch octenyl succinate, from Akzo Nobel

### Examples 25-31

The compositions used in Examples 25 to 31 are generally prepared in the same manner used for the compositions of Examples 11-17. Table 6 shows the ingredients used to make Examples 25-31, which utilize a combination of starch and silicone elastomer powder systems.

TABLE 6

| | Ex. 25 | Ex. 26 | Ex. 27 | Ex. 28 | Ex. 29 | Ex. 30 | Ex.31 |
|---|---|---|---|---|---|---|---|
| Water Phase: | | | | | | | |
| Water | qs | qs | qs | qs | qs | Qs | qs |
| Disodium EDTA | 0.1 | 0.05 | 0.05 | 0.1 | 0.05 | 0.05 | 0.05 |
| Benzyl Alcohol | 0.5 | 0.5 | — | 0.5 | — | — | — |
| Methylparaben | 0.25 | 0.25 | — | 0.2 | — | — | — |
| Iodopropynyl Butylcarbamate | 0.09 | 0.09 | 0.09 | 0.09 | 0.09 | 0.09 | 0.09 |
| Phenylbenzimidazole Sulfonic Acid | 1.0 | — | — | 2.0 | — | — | — |
| Symdiol 68¹ | — | — | 0.7 | — | 0.7 | 0.7 | 0.7 |
| Phenoxyethanol | — | — | 0.5 | — | 0.5 | 0.5 | 0.5 |
| Oil Phase: | | | | | | | |
| Isopropyl Isostearate | 2.0 | 0.5 | — | 2.0 | — | — | — |
| Isopropyl lauroylsarcosinate | — | — | — | 3.0 | — | — | — |
| Octisalate | 4.0 | 4.5 | — | — | — | — | — |
| Homosalate | — | 4.0 | — | 8.0 | — | — | — |
| Octocrylene | 1.0 | 2.6 | — | 2.25 | — | — | — |
| Octinoxate | — | — | 4.0 | — | 4.0 | 7.5 | 4.0 |
| Oxybenzone | — | — | — | — | — | — | — |
| Avobenzone | 2.0 | 3.0 | 2.0 | 3.0 | 2.0 | 3.0 | 2.0 |
| Solastay S1² | — | — | 0.5 | — | — | 1.0 | — |
| Stearic Acid | 0.1 | 0.1 | 0.05 | 0.05 | 0.05 | 0.05 | — |
| Ethylparaben | 0.2 | 0.2 | — | 0.2 | — | — | — |
| Propylparaben | 0.15 | 0.15 | — | 0.15 | — | — | — |
| Cetyl alcohol | 0.5 | 0.5 | 0.3 | 0.4 | 0.3 | 0.3 | 0.3 |
| Stearyl alcohol | 0.5 | 0.5 | 0.4 | 0.4 | 0.4 | 0.4 | 0.4 |
| Behenyl alcohol | 0.5 | 0.5 | 0.4 | 0.3 | 0.4 | 0.4 | 0.4 |
| Cetearyl Glucoside | 0.4 | 0.4 | 0.2 | 0.2 | 0.2 | 0.2 | 0.2 |
| PEG-100 stearate | 0.2 | 0.3 | 0.2 | 0.2 | 0.2 | 0.2 | 0.2 |
| Thickener: | | | | | | | |
| Xanthan Gum | — | — | 0.1 | — | — | — | — |
| Carbopol Ultrez 10³ | 0.2 | — | — | 0.2 | — | — | — |
| Simulgel INS-100⁴ | — | 1.0 | — | — | — | — | — |
| Sepigel 305⁵ | 1.0 | — | — | — | — | — | — |
| Makimousse-12⁶ | — | — | 0.4 | — | 0.4 | 0.4 | 0.4 |
| Simulgel EG⁷ | — | — | — | 2.25 | — | — | — |
| Powders: | | | | | | | |
| Dry Flo TS⁸ | — | — | 5.0 | 10.0 | — | 17.0 | — |
| Tapioca Pure⁹ | 10.0 | — | — | — | 10.0 | — | — |
| Dry Flo Pure¹⁰ | — | 10.0 | — | — | — | — | 10.0 |
| KSP 100¹¹ | — | 10.0 | — | — | 10.0 | 6.0 | — |
| KSP 101¹² | — | — | — | 6.0 | — | — | — |
| KSP 102¹³ | — | — | — | — | — | — | 1.0 |
| KSP 103¹⁴ | 5.0 | — | — | 4.0 | — | — | — |
| KSP 105¹⁵ | — | — | 5.0 | — | — | — | 1.0 |
| DC 9506¹⁶ | — | 5.0 | — | — | — | — | 3.0 |
| Total: | 100% | 100% | 100% | 100% | 100% | 100% | 100% |

¹1,2-hexanediol and caprylyl glycol, from Symrise
²Ethylhexyl Methoxycrylene, from Hallstar
³Carbomer, from Lubrizol
⁴Sodium acrylate/sodium acryloyldimethyl taurate copolymer, isohexadecane, and polysorbate 80, from Seppic
⁵Polyacrylamide, C13-14 isoparaffin, and laureth-7, from Seppic
⁶Sodium polyacrylate starch, from Kobo Products Inc.
⁷Sodium Acrylate/Sodium Acryloyldimethyl Taurate Copolymer & Isohexadecane & Polysorbate 80, from Seppic
⁸Tapioca and polymethylsilsesquioxane, from Akzo Nobel
⁹Tapioca powder, from Akzo Nobel
¹⁰Aluminum Starch octenyl succinate, from Akzo Nobel
¹¹Vinyl dimethicone/methicone silsesquioxane crosspolymer, from Shin Etsu
¹²Vinyl dimethicone/methicone silsesquioxane crosspolymer, from Shin Etsu
¹³Vinyl dimethicone/methicone silsesquioxane crosspolymer, from Shin Etsu
¹⁴Vinyl dimethicone/methicone silsesquioxane crosspolymer, from Shin Etsu
¹⁵Vinyl dimethicone/methicone silsesquioxane crosspolymer, from Shin Etsu
¹⁶Dimethicone/vinyldimethicone crosspolymer, from Dow Corning

### Comparative Examples 32-35

### Improving the Appearance of a Facial Moisturizer

The compositions in Examples 32-35 are generally prepared in the same manner described in examples 11-17. Table 7 shows the ingredients used in the compositions of Examples 32-35.

TABLE 7

| | 32 | 33 | 34 | 35 |
|---|---|---|---|---|
| | Finisher A | Finisher A | Finisher B | Finisher B |
| | with 15% | with 20% | with 15% | with 20% |
| | Elastomer | Starch | Elastomer | Starch |
| Water Phase: | | | | |
| Water | 74.224 | 69.224 | 70.33 | 65.33 |
| Disodium EDTA | 0.05 | 0.05 | 0.1 | 0.1 |
| Benzyl Alcohol | — | — | 0.5 | 0.5 |
| Methylparaben | — | — | 0.25 | 0.25 |
| Iodopropynyl | — | — | 0.09 | 0.09 |
| Butylcarbamate | | | | |
| Phenylbenzimidazole | — | — | 1.0 | 1.0 |
| Sulfonic Acid | | | | |
| Symdiol 68¹ | 0.8 | 0.8 | — | — |
| Phenoxyethanol | 0.376 | 0.376 | — | — |
| Oil Phase: | | | | |
| Isopropyl Isostearate | — | — | 1.33 | 1.33 |
| Octisalate | — | — | 4.0 | 4.0 |
| Octocrylene | — | — | 1.0 | 1.0 |
| Octinoxate | 4.0 | 4.0 | — | — |
| Avobenzone | 2.0 | 2.0 | 2.0 | 2.0 |
| Solastay S1² | 0.5 | 0.5 | — | — |
| Stearic Acid | 0.05 | 0.05 | 0.05 | 0.05 |
| Ethylparaben | — | — | 0.2 | 0.2 |
| Propylparaben | — | — | 0.15 | 0.15 |
| Cetyl alcohol | 0.3 | 0.3 | 0.3 | 0.3 |
| Stearyl alcohol | 0.4 | 0.4 | 0.4 | 0.4 |
| Behenyl alcohol | 0.4 | 0.4 | 0.4 | 0.4 |
| Cetearyl Glucoside | 0.2 | 0.2 | 0.2 | 0.2 |
| PEG-100 stearate | 0.2 | 0.2 | 0.1 | 0.1 |
| Thickener: | | | | |
| Carbopol Ultrez 10³ | — | — | 0.2 | 0.2 |
| Simulgel INS-100⁴ | 1.5 | 1.5 | 1.5 | 1.5 |
| pH Adjustor: | | | | |
| Triethanolamine | — | — | 0.9 | 0.9 |
| Powders: | | | | |
| Dry Flo TS⁵ | | 20.0 | | 20.0 |
| KSP 100⁶ | 15.0 | | 15.0 | |
| Total: | 100% | 100% | 100% | 100% |

¹1,2-hexanediol and caprylyl glycol, from Symrise
²Ethylhexyl Methoxycrylene, from Hallstar
³Carbomer, from Lubrizol
⁴Sodium acrylate/sodium acryloyldimethyl taurate copolymer, isohexadecane, and polysorbate 80, from Seppic
⁵Tapioca and polymethylsilsesquioxane, from Akzo Nobel
⁶Vinyl dimethicone/methicone silsesquioxane crosspolymer, from Shin Etsu

The compositions of Examples 32-35 were then placed in a Visual Attribute Test (“VAT”) to compare their effectiveness at reducing the appearance of various undesirable facial attributes. All of the examples were tested on top of Olay® Micro-Sculpting® brand facial moisturizing cream available from the Procter & Gamble Company, Cincinnati, Ohio. As can be seen from the results of this testing, summarized in Table 8 below, using the compositions of examples 32-35 on top of the facial moisturizer reduced the appearance attributes compared to using the facial moisturizer alone. Larger, positive VAT scores for bumpy surface and shine correspond to bigger visible reductions in the appearance of these attributes. Negative values indicate an increase in shine and bumpy surface.

TABLE 8

| | Step 1 - FM | Step 1 - FM | Step 1 - FM | Step 1 - FM | |
|---|---|---|---|---|---|
| | Step 2 - Finisher A | Step 2 - Finisher A | Step 2 - Finisher B | Step 2 - Finisher B | FM |
| | with 15% Elastomer | with 20% Starch | with 15% Elastomer | with 20% Starch | Alone |
| Bumpy | 0.23 | 0.27 | 0.23 | 0.25 | 0.06 |
| Surface | | | | | |
| Shine | 0.18 | −0.05 | 0.15 | 0.03 | −0.50 |

### Comparative Examples 36-39

### Impact of Powders in UV Finishers

The compositions in Examples 36-39 are generally prepared in the same manner as the composition of Examples 11-17. Table 9 shows the ingredients used in the compositions of Examples 36-39.

TABLE 9

| | 36 | 37 | 38 | 39 |
|---|---|---|---|---|
| | Finisher C | Finisher A | Finisher B | Finisher C |
| | with 20% | with No | with No | with No |
| | Starch | Powder | Powder | Powder |
| Water Phase: | | | | |
| Water | 64.224 | 89.224 | 85.33 | 84.224 |
| Disodium EDTA | 0.05 | 0.05 | 0.1 | 0.05 |
| Benzyl Alcohol | — | — | 0.5 | — |
| Methylparaben | — | — | 0.25 | — |
| Iodopropynyl | — | — | 0.09 | — |
| Butylcarbamate | | | | |
| Phenylbenzimidazole | — | — | 1.0 | — |
| Sulfonic Acid | | | | |
| Symdiol 68¹ | 0.8 | 0.8 | — | 0.8 |
| Phenoxyethanol | 0.376 | 0.376 | — | 0.376 |
| Oil Phase: | | | | |
| Isopropyl Isostearate | — | — | 1.33 | — |
| Octisalate | — | — | 4.0 | — |
| Octocrylene | — | — | 1.0 | — |
| Octinoxate | 4.0 | 4.0 | — | 7.5 |
| Avobenzone | 2.0 | 2.0 | 2.0 | 3.0 |
| Solastay S1² | 0.5 | 0.5 | — | 1.0 |
| Stearic Acid | 0.05 | 0.05 | 0.05 | 0.05 |
| Ethylparaben | — | — | 0.2 | — |
| Propylparaben | — | — | 0.15 | — |
| Cetyl alcohol | 0.3 | 0.3 | 0.3 | 0.3 |
| Stearyl alcohol | 0.4 | 0.4 | 0.4 | 0.4 |
| Behenyl alcohol | 0.4 | 0.4 | 0.4 | 0.4 |
| Cetearyl Glucoside | 0.2 | 0.2 | 0.2 | 0.2 |
| PEG-100 stearate | 0.2 | 0.2 | 0.1 | 0.2 |
| Thickener: | | | | |
| Carbopol Ultrez 10³ | — | — | 0.2 | — |
| Simulgel INS-100⁴ | 1.5 | 1.5 | 1.5 | 1.5 |
| pH Adjustor: | | | | |
| Triethanolamine | — | — | 0.9 | — |
| Powders: | | | | |
| Dry Flo TS⁵ | 20.0 | | | |
| Total: | 100% | 100% | 100% | 100% |

¹1,2-hexanediol and caprylyl glycol, from Symrise
²Ethylhexyl Methoxycrylene, from Hallstar
³Carbomer, from Lubrizol
⁴Sodium acrylate/sodium acryloyldimethyl taurate copolymer, isohexadecane, and polysorbate 80, from Seppic
⁵Tapioca and polymethylsilsesquioxane, from Akzo Nobel

The compositions of Examples 32-39 were then placed in a VAT to compare their effectiveness at reducing the appearance of various facial attributes. All of the examples were tested on top of Olay® brand facial moisturizer used in Examples 32-35. As can be seen from the results of this testing, summarized in Tables 10, 11, and 12 below, using the compositions containing the finisher compositions of Examples 32-36 in combination with the facial moisturizer (FM) reduced the undesirable appearance attributes relative to using the compositions that did not contain powders (examples 37-39).

TABLE 10

| | Step 1 - FM | Step 1 - FM | Step 1 - FM |
|---|---|---|---|
| | Step 2 - Finisher A | Step 2 - Finisher A | Step 2 - Finisher A |
| | with 15% Elastomer | with 20% Starch | with No Powder |
| | (Ex. 32) | (Ex. 33) | (Ex. 37) |
| Bumpy | 0.23 | 0.27 | −0.17 |
| Surface | | | |
| Shine | 0.18 | −0.05 | −1.10 |

TABLE 11

| | Step 1 - FM | Step 1 - FM | Step 1 - FM |
|---|---|---|---|
| | Step 2 - Finisher | Step 2 - Finisher | Step 2 - Finisher |
| | B with | B with | B with |
| | 15% Elastomer | 20% Starch | No Powder |
| | (Ex. 34) | (Ex. 35) | (Ex. 38) |
| Bumpy Surface | 0.23 | 0.25 | −0.13 |
| Shine | 0.15 | 0.03 | −1.33 |

TABLE 12

| | Step 1 - FM | Step 1 - FM |
|---|---|---|
| | Step 2 - Finisher C | Step 2 - Finisher C |
| | with 20% Starch | with No Powder |
| | (Ex. 36) | (Ex. 39) |
| Bumpy Surface | 0.16 | −0.26 |
| Shine | −0.06 | −1.68 |

### Comparative Examples 40 and 41

### Impact of Glycerin

Examples 40 and 41 demonstrate the undesirable effect of high levels of humectant on the appearance attributes of a skin care product. The compositions in Examples 40 and 41 contain the same high level of silicone elastomer particles and non-volatile silicone oil, and are otherwise identical except that Example 40 contains 5% glycerin while Example 41 contains 25% glycerin.

The compositions in Examples 40 and 41 are prepared by first combining the water phase ingredients and thickener in a container and mixing until uniform. The oil phase ingredients are combined in a separate container and mixed until uniform. The particulates are next added to the oil phase and the combination is mixed until uniform. Finally, the oil/particulate phase is added to the water phase and the resulting emulsion is subjected to high shear mixing (e.g., Flacktek Speedmixer, or rotor-stator mill) Table 13 shows the ingredients used in the compositions of Examples 40 and 41.

TABLE 13

| | Example | Example |
|---|---|---|
| | 40 | 41 |
| Water Phase: | | |
| Water | 20.84 | 0.84 |
| Glycerin | 5.0 | 25.0 |
| Disodium EDTA | 0.05 | 0.05 |
| Glydant Plus Liquid¹ | 0.3 | 0.3 |
| Niacinamide | 2.0 | 2.0 |
| D-panthenol | 0.5 | 0.5 |
| Laureth-4 | 0.2 | 0.2 |
| Thickener: | | |
| Simulgel INS-100² | 2.0 | 2.0 |
| Oil Phase: | | |
| Cyclomethicone D5 | 24.2 | 22.42 |
| Dimethicone 50 cst | 4.39 | 4.39 |
| DC9045³ | 11.0 | 11.0 |
| Isopropyl lauroyl | 7.32 | 7.32 |
| sarcosinate | | |
| Polysorbate 60 | 0.2 | 0.2 |
| Particles: | | |
| KSP 102⁴ | 11.0 | 11.0 |
| KSP 105⁵ | 11.0 | 11.0 |
| Total: | 100% | 100% |

¹DMDM Hydantoin, Butane-1,3-diol, iodopropynyl butylcarbamate, water, from Lonza
²Sodium acrylate/sodium acryloyldimethyl taurate copolymer, isohexadecane, and polysorbate 60, from Seppic
³Cyclopentasiloxane and dimethicone crosspolymer, from Dow Corning
⁴Vinyl dimethicone/methicone silsesquioxane crosspolymer, from Shin Etsu
⁵Vinyl dimethicone/methicone silsesquioxane crosspolymer, from Shin Etsu

The compositions of Examples 40 and 41 were then placed in a VAT to compare their effectiveness at reducing the appearance of various facial attributes. As can be seen from the results of this testing, summarized in Table 14 below, increasing the level of glycerin from 5% to 25% significantly reduced the appearance attributes of these products in use. Larger VAT scores for bumpy surface and shine correspond to bigger visible reductions in the appearance of these attributes. Thus, these data clearly demonstrate the negative impact that high levels of glycerin can have on the appearance benefits of a skin care product.

TABLE 14

| | Example 40 | Example 41 |
|---|---|---|
| | 5% Glycerin | 25% Glycerin |
| Bumpy Surface | 0.29 | 0.09 |
| Shine | 0.23 | −0.16 |

### Comparative Examples 42, 43 and 44

### Impact of Layers

Examples 42, 43 and 44 compare the effects of combining a moisturizing composition (i.e., conventional skin care product) with a particulate composition in an “all-in-one” composition versus applying the present finisher composition as a standalone product to an underlying layer of a skin care composition in a two-step process.

Example 44 utilizes a novel finisher composition with a silicone elastomer powder system. The finisher composition is prepared by first combining the water phase ingredients and thickener in a container and mixing until uniform. The oil phase ingredients are combined in a separate container and mixed until uniform. The particulates are next added to the oil phase and the combination is mixed until uniform. Finally, the oil/particulate phase is added to the water phase and the resulting emulsion is subjected to high shear mixing (e.g., Flacktek Speedmixer, or rotor-stator mill). The “all-in-one” composition utilized in Example 42 is prepared in generally the same way as the compositions of examples 40 and 41, and the skin care composition of Example 43 is prepared in generally the same way as the compositions of Examples 1-10. The ingredients of the compositions of Example 42, 43 and 44 are shown below in Table 15.

TABLE 15

| | | Example | Example |
|---|---|---|---|
| | Example | 43 | 44 |
| | 42 | First Layer | Inventive |
| | All-in-One | No Powder | Finisher |
| Water Phase: | | | |
| Water | 28.91 | 76.685 | 47.91 |
| Glycerin | 15.0 | 15.0 | — |
| Disodium EDTA | 0.05 | 0.025 | 0.05 |
| Symdiol 68¹ | 0.7 | 0.7 | 0.7 |
| Glycacil L² | 0.09 | 0.09 | 0.09 |
| Niacinamide | 5.0 | 5.0 | — |
| D-panthenol | 0.5 | 0.5 | — |
| Thickener: | | | |
| Simulgel INS-100³ | 1.2 | 2.0 | 1.2 |
| Oil Phase: | | | |
| Cyclomethicone | 24.0 | — | 20.0 |
| D5 | | | |
| Dimethicone 50 cst | 3.75 | — | 3.75 |
| DC9045⁴ | 5.5 | — | 11.0 |
| Laureth-4 | 0.3 | — | 0.3 |
| Particles: | | | |
| DC Elastomer⁵ | 15.0 | — | 15.0 |
| Total: | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³Sodium acrylate/sodium acryloyldimethyl taurate copolymer, isohexadecane, and polysorbate 60, from Seppic
⁴Cyclopentasiloxane and dimethicone crosspolymer, from Dow Corning
⁵Spherical silicone elastomer powder, from Dow Corning, mean particle size of 40 μm and mean hardness of 40 A

The skin care composition in Example 42 is an all-in-one formulation that contains the same glycerin and skin active levels as the skin care composition of Example 43. The composition of Example 42 also includes the same powder system as Example 44.

The all-in-one composition above (Example 42) and the corresponding two step composition (Example 42+43) were placed in a VAT to compare their effectiveness at reducing the appearance of various facial attributes. The results of this testing are shown below in Table 16. As can be seen in Table 16, the two step system provides greater visible benefits than their corresponding all-in-one system, despite both systems using the same spherical silicone elastomer powder and non-volatile silicones. Thus, these results demonstrate the benefits of applying the present finisher over an underlying layer of a skin care product.

TABLE 16

| | All-in-One | Two-Step |
|---|---|---|
| | Example 42 | Examples 43 + 44 |
| Bumpy Surface | 0.20 | 0.42 |
| Shine | −0.04 | 0.08 |

### Comparative Examples 45, 46 and 47

### Impact of Non-Volatile Oil to Powder Ratio

The finisher compositions in Examples 45, 46 and 47 utilize a starch powder system that contains 20% starch particles and 50 cst dimethicone. While a starch particle system is used in these examples, it is believed that the silicone elastomer powder system of the present invention will generally provide the same results. More importantly, these examples demonstrate the importance of providing a suitable non-volatile oil to powder ratio. A key difference between Examples 45, 46 and 47 is the non-volatile oil to powder ratio. A few very minor adjustments were made to these formulations to ensure that they had similar physical properties and stability, but these adjustments are not expected to impact product performance. Examples 45, 46 and 47 are prepared in substantially the same manner as described above with regard to Example 44. Table 17 shows the ingredients used in Examples 45, 46, and 47 along with the ingredients used to make the underlying base layer for this test.

TABLE 17

| | Base Layer | Example | Example | Example |
|---|---|---|---|---|
| | Composition | 45 | 46 | 47 |
| Water Phase: | | | | |
| Water | 76.685 | 49.36 | 44.36 | 40.56 |
| Disodium EDTA | 0.025 | .05 | 0.05 | 0.05 |
| Glycerin | 15.0 | — | — | — |
| Symdiol 68¹ | 0.7 | 0.7 | 0.7 | 0.7 |
| Glycacil L² | 0.09 | 0.09 | 0.09 | 0.09 |
| Niacinamide | 5.0 | — | — | — |
| D-panthenol | 0.5 | — | — | — |
| Thickener: | | | | |
| Simulgel INS-100³ | 2.0 | 1.5 | 1.5 | 1.3 |
| Oil Phase: | — | — | — | — |
| Cyclomethicone D5 | — | 12.0 | 12.0 | 6.0 |
| Dimethicone 50 cst | — | 5.0 | 10.0 | 20.0 |
| DC9045⁴ | — | 11.0 | 11.0 | 11.0 |
| Laureth-4 | — | 0.3 | 0.3 | 0.3 |
| Particles: | | | | |
| Dry Flo TS⁵ | — | 20.0 | 20.0 | 20.0 |
| Total: | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³Sodium acrylate/sodium acryloyldimethyl taurate copolymer, isohexadecane, and polysorbate 60, from Seppic
⁴Cyclopentasiloxane and dimethicone crosspolymer, from Dow Corning
⁵Tapioca and polymethylsilsesquioxane, from Akzo Nobel

The compositions in Examples 45, 46, and 47 were placed in a VAT study as part of a two-step process, using the base layer in Table 17 as the underlying skin care product for each test. As can be seen from the results of this testing, summarized in Table 18 below, as the non-volatile oil-to-powder ratio increased, the appearance attributes provided by these compositions worsened. Thus, this data clearly shows the benefit of the preferred non-volatile oil to powder ratio in the finisher compositions herein.

TABLE 18

| | Base Layer + | Base Layer + | Base Layer + |
|---|---|---|---|
| | Example 45 | Example 46 | Example 47 |
| Non-Volatile Oil | 1:4 | 1:2 | 1:1 |
| to Powder Ratio | | | |
| Bumpy Surface | 0.67 | 0.58 | 0.33 |
| Shine | 0.15 | 0.08 | −0.05 |

### Comparative Example 48

### Impact of Order of Layers

This example demonstrates the importance of applying a finisher as an overlying layer to an underlying layer of a skin care product, and not the other way around. The composition used in Example 48 is made by first combining the water phase ingredients and mixing until uniform. Next, the thickener is added and the composition is again mixed until uniform. Table 19 shows the ingredients used to make the composition in Example 48.

TABLE 19

| | Example 48 |
|---|---|
| Water Phase: | |
| Water | 78.16 |
| Glycerin | 15.0 |
| Disodium EDTA | 0.05 |
| Symdiol 68¹ | 0.7 |
| Glycacil L² | 0.09 |
| Niacinamide | 5.0 |
| D-panthenol | 0.5 |
| Thickener: | |
| Makimousse-12³ | 0.5 |
| Total: | 100% |

¹1,2-hexanediol and caprylyl glycol, from Symrise
²Iodopropynyl butylcarbamate, PEG-4 laurate, PEG-4 dilaurate, and polyethylene glycol, from Lonza
³Sodium polyacrylate starch, from Kobo Products Inc.

Using the composition in Example 48 along with the base layer composition shown in Table 17 and the finisher of Example 45, a VAT study was conducted to understand the impact of the order in which the layers of the two step systems are applied to skin. Table 20 shows the two step systems placed in the VAT study. The intended order shown in Table 20 refers to the order wherein the skin care product is placed first, as the underlying layer followed by placement of the finisher as the overlying layer. The reverse order refers to placement of the finisher first as the underlying layer, followed by placement of the skin care product as the overlying layer. Note that the base layer composition from Table 17 and the composition of Example 48 differ from one another only in the thickener used, and this difference is not expected to have a significant impact on the optical benefit demonstrated in this test.

TABLE 20

| | Underlying Layer | Overlying Layer |
|---|---|---|
| Intended Order | Base Layer from Table 17 | Example 45 |
| Reverse Order | Example 45 | Example 48 |

As can be seen from the VAT results in Table 21 below, applying the finisher as an overlying layer as intended delivers suitable benefits for the various visible attributes tested. However, applying the finisher as the underlying layer provided significantly less visible benefit. Thus, these results confirm the importance of the order of application steps of the current invention.

TABLE 21

| | Reversed Order | Intended Order |
|---|---|---|
| Bumpy Surface | 0.28 | 0.67 |
| Shine | 0.00 | 0.15 |

### Comparative Examples 49 and 50

### Impact of Increased Opacity

The following two examples both contain the same high level of silicone elastomer spherical particles and non-volatile silicone oil, and are identical except that Example 50 contains 3.43% pigments while Example 49 does not contain pigments. The pigments used in Example 50 resulted in increased opacity compared to Example 49. Opacity is assessed by measuring contrast ratio (the higher the contrast ratio, the higher the level of opacity). Example 50 has a contrast ratio of 34, while example 49 has a contrast ratio of 4.3. Examples 49 and 50 are prepared using the same process used for examples 40 and 41 above. Table 22 below shows the ingredients used to make the compositions for Examples 49 and 50.

TABLE 22

| | Example | Example |
|---|---|---|
| | 49 | 50 |
| Water Phase: | | |
| Water | 21.04 | 21.04 |
| Glycerin | 5.0 | 5.0 |
| Disodium EDTA | 0.05 | 0.05 |
| Glydant Plus Liquid¹ | 0.3 | 0.3 |
| Niacinamide | 2.0 | 2.0 |
| D-panthenol | 0.5 | 0.5 |
| Thickener: | | |
| Simulgel INS-100² | 2.0 | 2.0 |
| Oil Phase: | | |
| Cyclomethicone D5 | 24.2 | 20.77 |
| Dimethicone 50 cst | 4.39 | 4.39 |
| DC9045³ | 11.0 | 11.0 |
| Isononyl Isononanoate | 7.32 | 7.32 |
| Laureth-4 | 0.2 | 0.2 |
| Powders: | | |
| KSP 102⁴ | 11.0 | 11.0 |
| KSP 105⁵ | 11.0 | 11.0 |
| Pigments: | | |
| Titanium Dioxide⁶ | — | 3.0 |
| Iron Oxides CI 77491⁷ | — | 0.1 |
| Iron Oxides CI 77492⁸ | — | 0.33 |
| Total: | 100% | 100% |

¹DMDM Hydantoin, Butane-1,3-diol, iodopropynyl butylcarbamate, water, from Lonza
²Sodium acrylate/sodium acryloyldimethyl taurate copolymer, isohexadecane, and polysorbate 60, from Seppic
³Cyclopentasiloxane and dimethicone crosspolymer, from Dow Corning
⁴Vinyl dimethicone/methicone silsesquioxane crosspolymer, from Shin Etsu
⁵Vinyl dimethicone/methicone silsesquioxane crosspolymer, from Shin Etsu
⁶Titanium Dioxide, Isohexadecane, Polyhydroxystearic Acid, Triethoxycaprylylsilane
⁷Iron Oxides CI 77491, Cyclopentasiloxane, Methicone, PEG/PGG-18/18 Dimethicone
⁸Iron Oxides CI 77492, Cyclopentasiloxane, Methicone, PEG/PPG-18/18 Dimethicone

The Example 49 and 50 compositions were then placed in a VAT to compare their effectiveness at reducing the appearance of various facial attributes. Note that shine was not measured in this VAT, hence no shine results were reported. As can be seen from the results of this testing, summarized in Table 23 below, increasing opacity (higher contrast ratio) by using high refractive index pigments significantly reduced the optical benefit provided by the elastomer powder and silicone oil combination in these products. Thus, this data clearly demonstrates the negative impact that increased opacity has on the optical benefits of the powder and oil systems of the present invention.

TABLE 23

| | Example 49 | Example 50 |
|---|---|---|
| | 0% Pigment | 3.43% Pigment |
| | Contrast Ratio = 4.3 | Contrast Ratio = 34 |
| Bumpy Surface | 0.65 | −0.03 |

### Comparative Examples 51, 52, 53, and 54

### Particle Amount

This example demonstrates the importance of including the appropriate amount of powder in the finisher. The compositions used in Example 51 to 54 are prepared in substantially the same way as described previously for these types of compositions. Table 24 shows the ingredients used to make the composition in Examples 51 to 54. While Examples 52, 53 and 54 include starch particles, it is believed that spherical silicone elastomer particles would yield substantially the same results.

TABLE 24

| | Example | Example | Example | Example |
|---|---|---|---|---|
| | 51 | 52 | 53 | 54 |
| | First Layer | Finisher - | Finisher - | Finisher - |
| | No | 10% | 20% | 30% |
| | Powder | particles | particles | particles |
| Water Phase: | | | | |
| Water | 76.685 | 65.46 | 49.36 | 40.86 |
| Glycerin | 15.0 | | | |
| Disodium EDTA | 0.025 | 0.05 | 0.05 | 0.05 |
| Symdiol 68¹ | 0.7 | 0.7 | 0.7 | 0.7 |
| Glycacil L² | 0.09 | 0.09 | 0.09 | 0.09 |
| Niacinamide | 5.0 | — | — | — |
| D-panthenol | 0.5 | — | — | — |
| Thickener: | | | | |
| Simulgel INS-100³ | 2.0 | 2.0 | 1.5 | 1.5 |
| Oil Phase: | | | | |
| Cyclomethicone | — | 8.0 | 12.0 | 8.0 |
| D5 | | | | |
| Dimethicone 50 cst | — | 2.5 | 5.0 | 7.5 |
| DC9045⁴ | — | 11.0 | 11.0 | 11.0 |
| Laureth-4 | — | 0.2 | 0.3 | 0.3 |
| Particles: | | | | |
| Dry Flo TS⁵ | — | 10.0 | 20.0 | 30.0 |
| Total: | 100% | 100% | 100% | 100% |

¹1,2-hexanediol and caprylyl glycol, from Symrise
²Iodopropynyl butylcarbamate, PEG-4 laurate, PEG-4 dilaurate, and polyethylene glycol, from Lonza
³Sodium acrylate/sodium acryloyldimethyl taurate copolymer, isohexadecane, and polysorbate 60, from Seppic
⁴Cyclopentasiloxane and dimethicone crosspolymer, from Dow Corning
⁵Tapioca and polymethylsilsesquioxane, from Akzo Nobel

Table 25 shows the test results from this testing. As seen in Table 25, the appearance benefit improves from 10% powder level to 20% powder level, but the worsens from 20% to 30% powder level.

TABLE 25

| | Average VAT Score | | |
|---|---|---|---|
| | 10% particles | 20% particles | 30% particles |
| Bumpy Surface | 0.43 | 0.67 | 0.52 |
| Shine | 0.02 | 0.15 | 0.10 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of improving the look and feel of skin, comprising:
a. applying an underlying layer of a skin care composition to a target portion of skin; and
b. applying an overlying layer of a finisher composition on top of the underlying skin care composition layer, wherein the finisher composition is an oil-in-water emulsion comprising
i. a continuous aqueous phase that include from about 20 to 85% by weight of the finisher composition of water,
ii. a dispersed oil phase comprising a non-volatile oil, the non-volatile oil comprising a liquid UV agent present at an amount of at least 50% by weight of the non-volatile oil,
iii. from 10 to 25%, by weight of substantially spherical particles selected from the group consisting of starch particles, silicone elastomer particles and combinations thereof, wherein the particles have a particle size of from about 2 to 40 microns and a weight ratio of the non-volatile oil to the particles is from about 1:10 to about 1:1,
iv. optionally, from about 1 to 20 wt % of a volatile oil, and
v. wherein the finisher composition is substantially free of glycerin.

2. The finisher composition of claim 1, wherein the weight ratio of the non-volatile oil to the particles is from about 1:5 to about 4:5.

3. The finisher composition of claim 2, wherein the weight ratio of non-volatile oil to the particles is from about 1:4 to about 3:5

4. The finisher composition of claim 1, wherein the liquid UV agent is present at an amount of at least 90% by weight of the non-volatile oil.

5. The finisher composition of claim 4, wherein the non-volatile oil consists of the liquid UV agent.

6. The finisher composition of claim 1, where the liquid UV agent further comprises an oil soluble solid UV agent.

7. The finisher composition of claim 1, wherein the liquid UV agent comprises Ethylhexyl Dimethyl PABA, Ethylhexyl Methoxycinnamate (octinoxate), Ethylhexyl Salicylate (octisalate), Homosalate, Isoamyl p-Methoxycinnamate (amiloxate), Menthyl Anthranilate (meradimate), Octocrylene, Polysilicone-15 (diethylbenzylidene malonate dimethicone), Benzophenone-3 (oxybenzone), Benzophenone-9 (dioxybenzone), Bis-Ethylhexyloxyphenol Methoxyphenyl Triazine (bemotrizinol), Butyl Methoxydibenzoylmethane (avobenzone), Diethylamino Hydroxybenzoyl Hexyl Benzoate, Diethylhexyl Butamido Triazone (iscotrizinol), Drometrizole Trisiloxane, Ethylhexyl Triazone (octyl triazone), 4-Methylbenzylidene Camphor (enacamene) or a combination of these.

8. The finisher composition of claim 1, wherein the substantially spherical particles are silicone elastomer particles and the particles are present at an amount of from about 14 wt % to about 20 wt %.

9. The finisher composition of claim 1, wherein the substantially spherical particles are starch particles selected from the group consisting of tapioca starch, corn starch, potato starch, glyceryl starch, aluminum starch octenylsuccinate, calcium starch octenylsuccinate, polymethylsilsesquioxane coated tapioca starch, and combinations thereof.

10. The finisher composition of claim 9, wherein the substantially spherical starch particles have a mean particle size of from 5 to 30 microns.

11. The composition of claim 1, further comprising a chroma of less than about 10 according to the Chroma Method.

12. The composition of claim 1, further comprising a chroma of less than about 6 according to the Chroma Method.

13. The finisher composition of claim 1, wherein the composition is substantially free of humectants.

14. A method of improving the look and feel of skin, comprising:
a. applying an underlying layer of a skin care composition to a target portion of skin; and
b. applying an overlying layer of a finisher composition on top of the underlying skin care composition layer, wherein the finisher composition is an oil-in-water emulsion comprising
i. a continuous aqueous phase comprising from about 20 to 85 wt % of water,
ii. a dispersed oil phase comprising a non-volatile oil, wherein the non-volatile oil comprises a liquid UV agent present at an amount of at least 50% by weight of the non-volatile oil,
iii. from about 10 to 25 wt % of substantially spherical particles selected from the group consisting of starch particles, silicone elastomer particles, and combinations thereof, wherein the particles have a mean particle size of from about 2 to 40 microns and wherein a weight ratio of the non-volatile oil to the particles is from about 1:10 to about 1:1,
iv. less than 1 wt % of a pigment, and
v. a contrast ratio of less than about 20 according to the Contrast Ratio method.

15. The composition of claim 14, wherein the composition has a contrast ratio of less than about 10.

16. The composition of claim 14, wherein the composition is substantially free of pigments.

17. The composition of claim 14, wherein the composition includes less than 4% non-spherical particles.

18. The composition of claim 14, wherein the underlying layer of skin care composition comprises a humectant.

19. The composition of claim 18, wherein the humectant is glycerin.
